SEQUENCE LISTING (6 sequences)

SEQ ID NO: 1 - protein, 841 aa, Homo sapiens
MLLCTARLVGLQLLISCCWAFACHSTESSPDFTLPGDYLLAGLFPLHSGCLQVRHRPEVTLCDRSCSFNEHGYHLFQAMRLGVEEINNSTALLPNITLGYQLYDVCSDSANVYATLRVLSLPGQHHIELQGDLLHYSPTVLAVIGPDSTNRAATTAALLSPFLVPMISYAASSETLSVKRQYPSFLRTIPNDKYQVETMVLLLQKFGWTWISLVGSSDDYGQLGVQALENQATGQGICIAFKDIMPFSAQVGDERMQCLMRHLAQAGATVVVVFSSRQLARVFFESVVLTNLTGKVWVASEAWALSRHITGVPGIQRIGMVLGVAIQKRAVPGLKAFEEAYARADKKAPRPCHKGSWCSSNQLCRECQAFMAHTMPKLKAFSMSSAYNAYRAVYAVAHGLHQLLGCASGACSRGRVYPWQLLEQIHKVHFLLHKDTVAFNDNRDPLSSYNIIAWDWNGPKWTFTVLGSSTWSPVQLNINETKIQWHGKDNQVPKSVCSSDCLEGHQRVVTGFHHCCFECVPCGAGTFLNKSDLYRCQPCGKEEWAPEGSQTCFPRTVVFLALREHTSWVLLAANTLLLLLLLGTAGLFAWHLDTPVVRSAGGRLCFLMLGSLAAGSGSLYGFFGEPTRPACLLRQALFALGFTIFLSCLTVRSFQLIIIFKFSTKVPTFYHAWVQNHGAGLFVMISSAAQLLICLTWLVVWTPLPAREYQRFPHLVMLECTETNSLGFILAFLYNGLLSISAFACSYLGKDLPENYNEAKCVTFSLLFNFVSWIAFFTTASVYDGKYLPAANMMAGLSSLSSGFGGYFLPKCYVILCRPDLNSTEHFQASIQDYTRRCGST

SEQ ID NO: 2 - DNA, 2526 bp, Homo sapiens
  source 1..2526: mol_type = genomic DNA
atgctgctctgcacggctcgcctggtcggcctgcagcttctcatttcctgctgctgggcctttgcctgccatagcacggagtcttctcctgacttcaccctccccggagattacctcctggcaggcctgttccctctccattctggctgtctgcaggtgaggcacagacccgaggtgaccctgtgtgacaggtcttgtagcttcaatgagcatggctaccacctcttccaggctatgcggcttggggttgaggagataaacaactccacggccctgctgcccaacatcaccctggggtaccagctgtatgatgtgtgttctgactctgccaatgtgtatgccacgctgagagtgctctccctgccagggcaacaccacatagagctccaaggagaccttctccactattcccctacggtgctggcagtgattgggcctgacagcaccaaccgtgctgccaccacagccgccctgctgagccctttcctggtgcccatgattagctatgcggccagcagcgagacgctcagcgtgaagcggcagtatccctctttcctgcgcaccatccccaatgacaagtaccaggtggagaccatggtgctgctgctgcagaagttcgggtggacctggatctctctggttggcagcagtgacgactatgggcagctaggggtgcaggcactggagaaccaggccactggtcaggggatctgcattgctttcaaggacatcatgcccttctctgcccaggtgggcgatgagaggatgcagtgcctcatgcgccacctggcccaggccggggccaccgtcgtggttgttttttccagccggcagttggccagggtgtttttcgagtccgtggtgctgaccaacctgactggcaaggtgtgggtcgcctcagaagcctgggccctctccaggcacatcactggggtgcccgggatccagcgcattgggatggtgctgggcgtggccatccagaagagggctgtccctggcctgaaggcgtttgaagaagcctatgcccgggcagacaagaaggcccctaggccttgccacaagggctcctggtgcagcagcaatcagctctgcagagaatgccaagctttcatggcacacacgatgcccaagctcaaagccttctccatgagttctgcctacaacgcataccgggctgtgtatgcggtggcccatggcctccaccagctcctgggctgtgcctctggagcttgttccaggggccgagtctacccctggcagcttttggagcagatccacaaggtgcatttccttctacacaaggacactgtggcgtttaatgacaacagagatcccctcagtagctataacataattgcctgggactggaatggacccaagtggaccttcacggtcctcggttcctccacatggtctccagttcagctaaacataaatgagaccaaaatccagtggcacggaaaggacaaccaggtgcctaagtctgtgtgttccagcgactgtcttgaagggcaccagcgagtggttacgggtttccatcactgctgctttgagtgtgtgccctgtggggctgggaccttcctcaacaagagtgacctctacagatgccagccttgtgggaaagaagagtgggcacctgagggaagccagacctgcttcccgcgcactgtggtgtttttggctttgcgtgagcacacctcttgggtgctgctggcagctaacacgctgctgctgctgctgctgcttgggactgctggcctgtttgcctggcacctagacacccctgtggtgaggtcagcagggggccgcctgtgctttcttatgctgggctccctggcagcaggtagtggcagcctctatggcttctttggggaacccacaaggcctgcgtgcttgctacgccaggccctctttgcccttggtttcaccatcttcctgtcctgcctgacagttcgctcattccaactaatcatcatcttcaagttttccaccaaggtacctacattctaccacgcctgggtccaaaaccacggtgctggcctgtttgtgatgatcagctcagcggcccagctgcttatctgtctaacttggctggtggtgtggaccccactgcctgctagggaataccagcgcttcccccatctggtgatgcttgagtgcacagagaccaactccctgggcttcatactggccttcctctacaatggcctcctctccatcagtgcctttgcctgcagctacctgggtaaggacttgccagagaactacaacgaggccaaatgtgtcaccttcagcctgctcttcaacttcgtgtcctggatcgccttcttcaccacggccagcgtctacgacggcaagtacctgcctgcggccaacatgatggctgggctgagcagcctgagcagcggcttcggtgggtattttctgcctaagtgctacgtgatcctctgccgcccagacctcaacagcacagagcacttccaggcctccattcaggactacacgaggcgctgcggctccacctga

SEQ ID NO: 3 - protein, 857 aa, Rattus norvegicus
MPGLAILGLSLAAFLELGMGSSLCLSQQFKAQGDYILGGLFPLGTTEEATLNQRTQPNGILCTRFSPLGLFLAMAMKMAVEEINNGSALLPGLRLGYDLFDTCSEPVVTMKPSLMFMAKVGSQSIAAYCNYTQYQPRVLAVIGPHSSELALITGKFFSFFLMPQVSYSASMDRLSDRETFPSFFRTVPSDRVQLQAVVTLLQNFSWNWVAALGSDDDYGREGLSIFSGLANSRGICIAHEGLVPQHDTSGQQLGKVVDVLRQVNQSKVQVVVLFASARAVYSLFSYSILHDLSPKVWVASESWLTSDLVMTLPNIARVGTVLGFLQRGALLPEFSHYVETRLALAADPTFCASLKAELDLEERVMGPRCSQCDYIMLQNLSSGLMQNLSAGQLHHQIFATYAAVYSVAQALHNTLQCNVSHCHTSEPVQPWQLLENMYNMSFRARDLTLQFDAKGSVDMEYDLKMWVWQSPTPVLHTVGTFNGTLQLQHSKMYWPGNQVPVSQCSRQCKDGQVRRVKGFHSCCYDCVDCKAGSYRKHPDDFTCTPCGKDQWSPEKSTTCLPRRPKFLEWGEPAVLLLLLLLSLALGLVLAALGLFVHHRDSPLVQASGGPLACFGLVCLGLVCLSVLLFPGQPSPARCLAQQPLSHLPLTGCLSTLFLQAAEIFVESELPLSWADRLSGCLRGPWAWLVVLLAMLVEVALCTWYLVAFPPEVVTDWHMLPTEALVHCRTRSWVSFGLAHATNATLAFLCFLGTFLVRSQPGRYNRARGLTFAMLAYFITWVSFVPLLANVQVVLRPAVQMGALLLCVLGILAAFHLPRCYLLMRQPGLNTPEFFLGGGPGDAQGQNDGNTGNQGKHE

SEQ ID NO: 4 - DNA, 2574 bp, Rattus norvegicus
  source 1..2574: mol_type = genomic DNA
atgccgggtttggctatcttgggcctcagtctggctgctttcctggagcttgggatggggtcctctttgtgtctgtcacagcaattcaaggcacaaggggactatatattgggtggactatttcccctgggcacaactgaggaggccactctcaaccagagaacacagcccaacggcatcctatgtaccaggttctcgccccttggtttgttcctggccatggctatgaagatggctgtagaggagatcaacaatggatctgccttgctccctgggctgcgactgggctatgacctgtttgacacatgctcagagccagtggtcaccatgaagcccagcctcatgttcatggccaaggtgggaagtcaaagcattgctgcctactgcaactacacacagtaccaaccccgtgtgctggctgtcattggtccccactcatcagagcttgccctcattacaggcaagttcttcagcttcttcctcatgccacaggtcagctatagtgccagcatggatcggctaagtgaccgggaaacatttccatccttcttccgcacagtgcccagtgaccgggtgcagctgcaggccgttgtgacactgttgcagaatttcagctggaactgggtggctgccttaggtagtgatgatgactatggccgggaaggtctgagcatcttttctggtctggccaactcacgaggtatctgcattgcacacgagggcctggtgccacaacatgacactagtggccaacaattgggcaaggtggtggatgtgctacgccaagtgaaccaaagcaaagtacaggtggtggtgctgtttgcatctgcccgtgctgtctactccctttttagctacagcatccttcatgacctctcacccaaggtatgggtggccagtgagtcctggctgacctctgacctggtcatgacacttcccaatattgcccgtgtgggcactgttcttgggtttctgcagcgcggtgccctactgcctgaattttcccattatgtggagactcgccttgccctagctgctgacccaacattctgtgcctccctgaaagctgagttggatctggaggagcgcgtgatggggccacgctgttcacaatgtgactacatcatgctacagaacctgtcatctgggctgatgcagaacctatcagctgggcagttgcaccaccaaatatttgcaacctatgcagctgtgtacagtgtggctcaggcccttcacaacaccctgcagtgcaatgtctcacattgccacacatcagagcctgttcaaccctggcagctcctggagaacatgtacaatatgagtttccgtgctcgagacttgacactgcagtttgatgccaaagggagtgtagacatggaatatgacctgaagatgtgggtgtggcagagccctacacctgtactacatactgtaggcaccttcaacggcacccttcagctgcagcactcgaaaatgtattggccaggcaaccaggtgccagtctcccagtgctcccggcagtgcaaagatggccaggtgcgcagagtaaagggctttcattcctgctgctatgactgtgtggactgcaaggcagggagctaccggaagcatccagatgacttcacctgtactccatgtggcaaggatcagtggtccccagaaaaaagcacaacctgcttacctcgcaggcccaagtttctcgagtggggcgagccggctgtgctgctgctgctcctgctgctgagcctggcgctgggccttgtgctggctgctttggggctgttcgttcaccatcgggacagcccactggttcaggcctcgggggggcccctggcctgctttggcctggtgtgcctgggcctggtctgcctcagcgtcctcctgttccctggccagcccagccctgcccgatgcctggcccagcagcccttgtcccacctcccgctcacgggctgcctgagcacactcttcctgcaggcggccgagatcttcgtggagtcagaactgcctctgagctgggcagaccggctgagtggctgcctgcgggggccctgggcctggctggtggtgctgctggccatgctggtggaggtcgcactgtgcacctggtacctggtggccttcccgccggaggtggtgacggactggcacatgctgcccacggaggcgctggtgcactgccgcacacgctcctgggtcagcttcggcctagcgcacgccaccaatgccacgctggcctttctctgcttcctgggcactttcctggtgcggagccagccgggccgctacaaccgtgcccgtggcctcacctttgccatgctggcctacttcatcacctgggtctcctttgtgcccctcctggccaatgtgcaggtggtcctcaggcccgccgtgcagatgggcgccctcctgctctgtgtcctgggcatcctggctgccttccacctgcccaggtgttacctgctcatgcggcagccagggctcaacacccccgagttcttcctgggagggggccctggggatgcccaaggccagaatgacgggaacacaggaaatcaggggaaacatgagtga

SEQ ID NO: 5 - protein, 374 aa, Homo sapiens
MARSLTWRCCPWCLTEDEKAAARVDQEINRILLEQKKQDRGELKLLLLGPGESGKSTFIKQMRIIHGAGYSEEERKGFRPLVYQNIFVSMRAMIEAMERLQIPFSRPESKHHASLVMSQDPYKVTTFEKRYAAAMQWLWRDAGIRACYERRREFHLLDSAVYYLSHLERITEEGYVPTAQDVLRSRMPTTGINEYCFSVQKTNLRIVDVGGQKSERKKWIHCFENVIALIYLASLSEYDQCLEENNQENRMKESLALFGTILELPWFKSTSVILFLNKTDILEEKIPTSHLATYFPSFQGPKQDAEAAKRFILDMYTRMYTGCVDGPEGSKKGARSRRLFSHYTCATDTQNVKFVFDAVTDIIIKENLKDCGLF

SEQ ID NO: 6 - DNA, 1125 bp, Homo sapiens
  source 1..1125: mol_type = genomic DNA
atggcccgctcgctgacctggcgctgctgcccctggtgcctgacggaggatgagaaggccgccgcccgggtggaccaggagatcaacaggatcctcttggagcagaagaagcaggaccgcggggagctgaagctgctgcttttgggcccaggcgagagcgggaagagcaccttcatcaagcagatgcggatcatccacggcgccggctactcggaggaggagcgcaagggcttccggcccctggtctaccagaacatcttcgtgtccatgcgggccatgatcgaggccatggagcggctgcagattccattcagcaggcccgagagcaagcaccacgctagcctggtcatgagccaggacccctataaagtgaccacgtttgagaagcgctacgctgcggccatgcagtggctgtggagggatgccggcatccgggcctgctatgagcgtcggcgggaattccacctgctcgattcagccgtgtactacctgtcccacctggagcgcatcaccgaggagggctacgtccccacagctcaggacgtgctccgcagccgcatgcccaccactggcatcaacgagtactgcttctccgtgcagaaaaccaacctgcggatcgtggacgtcgggggccagaagtcagagcgtaagaaatggatccattgtttcgagaacgtgatcgccctcatctacctggcctcactgagtgaatacgaccagtgcctggaggagaacaaccaggagaaccgcatgaaggagagcctcgcattgtttgggactatcctggaactaccctggttcaaaagcacatccgtcatcctctttctcaacaaaaccgacatcctggaggagaaaatccccacctcccacctggctacctatttccccagtttccagggccctaagcaggatgctgaggcagccaagaggttcatcctggacatgtacacgaggatgtacaccgggtgcgtggacggccccgagggcagcaagaagggcgcacgatcccgacgccttttcagccactacacatgtgccacagacacacaaaacgtcaaattcgtgtttgatgccgtgacagatataataataaaagagaacctcaaagactgtgggctcttctga

(19) United States
(12) Patent Application Publication
(10) Pub. No.: US 2025/0155427 A1
(43) Pub. Date: May 15, 2025

(54) CELLS EXPRESSING UMAMI TASTE RECEPTORS AND USES THEREOF

(71) Applicant: Firmenich Incorporated, Plainsboro, NJ (US)

(72) Inventors: Guy Servant, Plainsboro, NJ (US); Mark Williams, Plainsboro, NJ (US)

(21) Appl. No.: 18/837,833

(22) PCT Filed: Jan. 13, 2023
(86) PCT No.: PCT/US2023/060652

§ 371 Date: Aug. 12, 2024

Related U.S. Application Data
(60) Provisional application No. 63/309,402, filed on Feb. 11, 2022.

(30) Foreign Application Priority Data
Apr. 25, 2022 (EP) 22169713.9

(51) Int. Cl. G01N 33/50; C07K 14/47; C07K 14/705; C12N 5/07; G01N 21/64

(52) U.S. Cl. CPC G01N 33/5041; C07K 14/4722; C07K 14/705; C12N 5/0601; G01N 21/6486; G01N 33/5044; G01N 2333/4719; G01N 2333/705

(57) ABSTRACT

The present disclosure generally relates to the expression of T1R1+3 taste receptors in eukaryotic cells, such as U2OS cells. The disclosure also relates to the expression of a chimeric T1R3 taste receptor subunit to enable the T1R1 taste receptor subunit to achieve a higher level of sensitivity to umami compounds. Also disclosed are methods of expressing T1R1+3 taste receptors in such cells, and methods of screening for substances that modulate T1R1+3 receptor sensing, and which, therefore, may be useful as modulators of umami taste.

### BACKGROUND

### Field

The present disclosure generally relates to the expression of T1R1 taste receptors in eukaryotic cells, such as U2OS cells. The disclosure also relates to methods of expressing T1R1 taste receptors in such cells, and to methods of screening for substances that modulate T1R1 receptors, and which, therefore, may be useful as modulators of umami taste.

### Background Description

The taste system provides sensory information about the chemical composition of the external world. Taste transduction is one of the most sophisticated forms of chemical-triggered sensation in animals. Signaling of taste is found throughout the animal kingdom, from simple metazoans to the most complex of vertebrates. Mammals are believed to have five basic taste modalities: sweet, bitter, sour, salty, and umami (the savory taste of monosodium glutamate). Numerous physiological studies in animals have shown that taste receptor cells may selectively respond to different chemical stimuli. See, e.g., Akabas et al., Science, 242:1047-50 (1988); Gilbertson et al., J. Gen. Physiol., 100:803-24 (1992); Bernhardt et al., J. Physiol., 490:325-36 (1996); Cummings et al., J. Neurophysiol., 75:1256-63 (1996).

Taste receptors specifically recognize molecules that elicit specific taste sensation. These molecules are also referred to herein as “tastants.” Many taste receptors belong to the 7-transmembrane receptor superfamily (Hoon et al., Cell 96:451 (1999); Adler et al., Cell 100:693 (2000)), which are also known as G-protein-coupled receptors (GPCRs). Other tastes are believed to be mediated by channel proteins. G-protein-coupled receptors control many physiological functions, such as endocrine function, exocrine function, heart rate, lipolysis, carbohydrate metabolism, and transmembrane signaling. The biochemical analysis and molecular cloning of a number of such receptors has revealed many basic principles regarding the function of these receptors.

In mammals, taste receptor cells are assembled into taste buds that are distributed into different papillae in the tongue epithelium. Circumvallate papillae, found at the very back of the tongue, contain hundreds to thousands of taste buds. By contrast, foliate papillae, localized to the posterior lateral edge of the tongue, contain dozens to hundreds of taste buds. Further, fungiform papillae, located at the front of the tongue, contain only a single or a few taste buds.

Each taste bud, depending on the species, contains 50-150 cells, including precursor cells, support cells, and taste receptor cells. See, e.g., Lindemann, Physiol. Rev., 76:718-66 (1996). Receptor cells are innervated at their base by afferent nerve endings that transmit information to the taste centers of the cortex through synapses in the brain stem and thalamus. Elucidating the mechanisms of taste cell signaling and information processing is important to understanding the function, regulation, and perception of the sense of taste.

### SUMMARY

The present disclosure describes expression of a chimeric umami taste receptor. Expression of the chimeric umami taste receptor may be achieved in, for example, U2OS cells. Also described is a method for screening potential umami taste receptor modulators. The chimeric umami taste receptor provides a broader signal window than a human umami receptor.

In one embodiment, a cell includes a human T1R1 umami taste receptor subunit, a rat-human T1R3 taste receptor subunit, and a G-protein, and where the cell is not an HEK cell. The cell may be a eukaryotic cell. The cell may be a U2OS cell. The G-protein may be coupled to the T1R1 umami taste receptor subunit. The T1R1 umami taste receptor subunit may include a polypeptide sequence at least 80% identical to a polypeptide sequence of SEQ ID NO. 1, or a functional fragment thereof. The T1R1 umami taste receptor subunit may include a polypeptide sequence at least 90% identical to a polypeptide sequence of SEQ ID NO. 1, or a functional fragment thereof. The T1R1 umami taste receptor subunit may include a polypeptide sequence at least 95% identical to a polypeptide sequence of SEQ ID NO. 1, or a functional fragment thereof. The T1R1 umami taste receptor subunit may include a polypeptide sequence identical to a polypeptide sequence of SEQ ID NO. 1, or a functional fragment thereof. The cell may include a polynucleotide sequence encoding for the human T1R1 taste receptor including a sequence at least 80% identical to a polynucleotide sequence of SEQ ID NO. 2, or a functional fragment thereof. The cell may include a polynucleotide sequence encoding for the human T1R1 taste receptor includes a sequence at least 90% identical to a polynucleotide sequence of SEQ ID NO. 2, or a functional fragment thereof. The cell may include a polynucleotide sequence encoding for the human T1R1 taste receptor includes a sequence at least 95% identical to a polynucleotide sequence of SEQ ID NO. 2, or a functional fragment thereof. The cell may include a polynucleotide sequence encoding for the human T1R1 taste receptor identical to a polynucleotide sequence of SEQ ID NO. 2, or a functional fragment thereof. The rat-human T1R3 taste receptor subunit may include a polypeptide sequence at least 80% identical to a polypeptide sequence of SEQ ID NO. 3, or a functional fragment thereof. The rat-human T1R3 taste receptor subunit may include a polypeptide sequence at least 90% identical to a polypeptide sequence of SEQ ID NO. 3, or a functional fragment thereof. The rat-human T1R3 taste receptor subunit may include a polypeptide sequence at least 95% identical to a polypeptide sequence of SEQ ID NO. 3, or a functional fragment thereof. The rat-human T1R3 taste receptor subunit may include a polypeptide sequence identical to a polypeptide sequence of SEQ ID NO. 3, or a functional fragment thereof. The G-protein may include any one of the following: a gustducin or a G(i/o) polypeptide. The G-protein may include a promiscuous G-protein. The G-protein may include a polypeptide sequence at least 90% identical to a polypeptide sequence of SEQ ID NO: 5 or a functional fragment thereof. The cell may include a polynucleotide sequence encoding for the rat-human T1R3 taste receptor subunit including a sequence at least 80% identical to a polynucleotide sequence of SEQ ID NO. 4, or a functional fragment thereof. The cell may include a polynucleotide sequence encoding for the rat-human T1R3 taste receptor subunit includes a sequence at least 90% identical to a polynucleotide sequence of SEQ ID NO. 4, or a functional fragment thereof. The cell may include a polynucleotide sequence encoding for the rat-human T1R3 taste receptor subunit includes a sequence at least 95% identical to a polynucleotide sequence of SEQ ID NO. 4, or a functional fragment thereof. The cell may include a polynucleotide sequence subunit encoding for the rat-human T1R3 taste receptor subunit identical to a polynucleotide sequence of SEQ ID NO. 4, or a functional fragment thereof. The cell may be modified to overexpress the T1R1 umami taste receptor subunit. The cell may be modified to overexpress the rat-human T1R3 taste receptor subunit. The cell may be modified to overexpress the G-protein. The cell may be an isolated cell. A cell-based assay may include the cell. A solid support may include the cell.

In one embodiment, a method of identifying an umami taste modulator, including contacting the cell with a test compound, and measuring a T1R1 umami taste receptor activity, where a change in the T1R1 umami taste receptor activity indicates that the test compound is an umami taste modulator. The promiscuous G-protein may include any one of the following: G15, G16, G16T44, or G16gust25. Measuring the T1R1 umami taste receptor activity may include detecting changes in intracellular calcium. Detecting changes in intracellular calcium may include detecting a Fluo-4 dependent fluorescence. The method may also include identifying the umami taste modulator as a compound that modulates umami taste. The measuring T1R1 umami taste receptor activity may include comparing the T1R1 umami taste receptor activity in presence of the test compound and in absence of the test compound. An increase in the T1R1 umami taste receptor activity may indicate that the test compound is an umami tastant. Measuring the T1R1 umami taste receptor activity may include comparing the T1R1 umami taste receptor activity in presence of a known umami compound and in absence of the known umami compound. A decrease in the T1R1 umami taste receptor activity may indicate that the test compound is an umami blocker. A method of altering umami taste of an ingestible composition, including an umami taste modulator identified by the method may also include in the ingestible composition. The ingestible composition may include a food product, a beverage product, a pharmaceutical product, or an oral care product. Other technical features may be readily apparent to one skilled in the art from the following figures, descriptions, and claims.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 schematically illustrates an example U2OS cell including a human T1R1 umami taste receptor subunit, a rat-human T1R3 taste receptor subunit, and a G-protein.

FIG. 2 schematically illustrates an example method for determining whether a test compound is an umami taste modulator.

FIG. 3 schematically illustrates an example method for determining whether a test compound is an umami taste modulator in the presence of a known umami tastant.

FIG. 4A illustrates dose-response data of a U2OS cell line expressing human T1R1, a rat-human T1R3, and a G-protein to MSG and umami enhancing compounds.

FIG. 4B illustrates dose-response data of a U2OS cell line expressing human T1R1, a rat-human T1R3, and a G-protein to umami modulating compounds UM33 and UM80.

### DETAILED DESCRIPTION

Aspects of the disclosure will herein be set forth in detail with respect to the figures and various examples. One of skill in the art will appreciate, however, that other configurations of the devices and methods disclosed herein will still fall within the scope of this disclosure even if not described in the same detail. Aspects of various configurations discussed do not limit the scope of the disclosure herein, which is instead defined by the claims following this description.

### Functional Expression of Taste Receptors

Taste receptors can be functionally expressed in cells. Such taste receptors are coupled to one or more G-proteins, as they would be in vivo. When a test molecule binds to the taste receptor, the coupled G-protein responds to the binding event by recruiting and activating intracellular enzymes and emitting a response, such as a change in the calcium cation concentration in the cell. Such chemical changes can be detected and measured, thereby providing a chemical signal indicative of the binding of the test molecule to the taste receptor. A plurality of cells expressing such G-protein-coupled taste receptors can be incorporated into a standard cellular assay. Using such assays, various taste compounds can be screened to determine the degree and manner in which they modulate taste receptor proteins. In such assays, cells may, for example, be disposed on a solid support such as a multi-well plate.

Taste receptor cells cannot be efficiently grown in the lab, but other cell types can be induced to overexpress a recombinant receptor along with a relevant G-protein. Cell type can have an effect on how robust such assays are. For example, most such assays have a signal window within which the detectable signal corresponds reasonably well with the activity of the test molecule with respect to the taste receptor. Outside of that signal window, the correspondence deteriorates, and the assay no longer provides useful information about the binding event.

### T1R1+3 Dimer

T1R1 is a G-protein-coupled receptor and a component of the heterodimeric amino acid taste receptor T1R1+3, which also includes receptor subunit T1R3. Generally, T1R1 is not functionally active unless it is in a T1R1+3 heterodimer with T1R3. T1R1 responds to umami compounds, and is thus an umami receptor.

As discussed previously, taste receptor cells cannot be efficiently grown in the lab, but other cell types can be induced to overexpress a recombinant receptor along with a relevant G-protein. U2OS cells, for example, can be maintained in culture and are amenable to recombinant gene expression, but activation of the human umami receptor T1R1+3 expressed in U2OS cells generates a very small calcium response.

However, replacement of the human T1R3 subunit within the T1R1+3 dimer with a rat T1R3 subunit or a rat-human T1R3 chimera may result in a functional umami receptor that generates a larger measurable response and adequate assay window for high-throughput screening. The rat-human T1R3 chimera may be preferable to the rat T1R3 subunit because the rat-human T1R3 subunit contains the human transmembrane domain, which may be more native in its interactions with the human T1R1 subunit.

### Cells Expressing T1R1 and T1R3 Receptors

Some embodiments include a cell expressing T1R1 and/or T1R3 umami taste receptors. In some embodiments, the cell is not an HEK cell. In some embodiments, the cell is a eukaryotic cell. In some embodiments, the cell is a U2OS cell. In certain related aspects, the disclosure provides a cell, such as a U2OS cell, that includes exogenous nucleic acid sequences that encode for a T1R1+3 umami taste receptor.

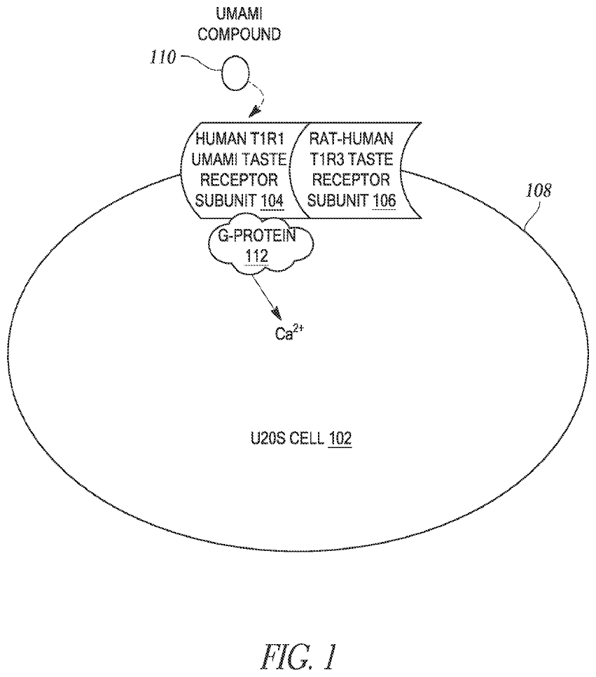

FIG. 1 schematically illustrates one embodiment. The modified cell includes a U2OS cell 102, a human T1R1 umami taste receptor subunit 104, a rat-human T1R3 taste receptor subunit 106, a coupled G-protein 112, and a cell membrane 108. The human T1R1 umami taste receptor subunit 104 and rat-human T1R3 taste receptor subunit 106 form a heterodimer that spans the cell membrane 108. Human T1R1 umami taste receptor subunit 104 is generally not functionally active unless it is dimerized with a T1R3 subunit such as rat-human T1R3 taste receptor subunit 106. When umami compound 110 binds to human T1R1 umami taste receptor subunit 104, coupled G-protein 112 increases intracellular Ca²⁺. The increase in intracellular Ca²⁺ may be detectable by assays well known in the art. For example, a Ca²⁺-sensitive fluorescent dye may optically indicate an increase in intracellular Ca²⁺. A Ca²⁺-sensitive fluorescent dye may be imaged to allow for measurement of T1R1 activity, as described in the art.

In some embodiments, the T1R1 umami taste receptor may be a human T1R1 umami taste receptor, or a functional fragment thereof.

In some embodiments, the T1R1 umami taste receptor subunit may be a polypeptide set forth as SEQ ID NO. 1, or a functional fragment thereof. SEQ ID NO. 1 is MLLCTARLVGLQLLISCCWAFACHSTESSPDFTLPGDYLLAGLFPLHSGCLQVRHRPEVT LCDRSCSFNEHGYHLFQAMRLGVEEINNSTALLPNITLGYQLYDVCSDSANVYATLRVL SLPGQHHIELQGDLLHYSPTVLAVIGPDSTNRAATTAALLSPFLVPMISYAASSETLSVKR QYPSFLRTIPNDKYQVETMVLLLQKFGWTWISLVGSSDDYGQLGVQALENQATGQGICI AFKDIMPFSAQVGDERMQCLMRHLAQAGATVVVVFSSRQLARVFFESVVLTNLTGKV WVASEAWALSRHITGVPGIQRIGMVLGVAIQKRAVPGLKAFEEAYARADKKAPRPCHK GSWCSSNQLCRECQAFMAHTMPKLKAFSMSSAYNAYRAVYAVAHGLHQLLGCASGAC SRGRVYPWQLLEQIHKVHFLLHKDTVAFNDNRDPLSSYNIIAWDWNGPKWTFTVLGSST WSPVQLNINETKIQWHGKDNQVPKSVCSSDCLEGHQRVVTGFHHCCFECVPCGAGTFL NKSDLYRCQPCGKEEWAPEGSQTCFPRTVVFLALREHTSWVLLAANTLLLLLLLGTAGL FAWHLDTPVVRSAGGRLCFLMLGSLAAGSGSLYGFFGEPTRPACLLRQALFALGFTIFLS CLTVRSFQLIIIFKFSTKVPTFYHAWVQNHGAGLFVMISSAAQLLICLTWLVVWTPLPAR EYQRFPHLVMLECTETNSLGFILAFLYNGLLSISAFACSYLGKDLPENYNEAKCVTFSLLF NFVSWIAFFTTASVYDGKYLPAANMMAGLSSLSSGFGGYFLPKCYVILCRPDLNSTEHF QASIQDYTRRCGST.

In some embodiments, the T1R1 umami taste receptor subunit may have a polypeptide sequence at least 70% similar to the sequence of SEQ ID NO. 1, at least 80% similar to the sequence of SEQ ID NO. 1, at least 90% similar to the sequence of SEQ ID NO. 1, at least 95% similar to the sequence of SEQ ID NO. 1, at least 96% similar to the sequence of SEQ ID NO. 1, at least 97% similar to the sequence of SEQ ID NO. 1, at least 98% similar to the sequence of SEQ ID NO. 1, at least 99% similar to the sequence of SEQ ID NO. 1, or identical to the sequence of SEQ ID NO. 1.

In some embodiments, the T1R1 polypeptide set forth as SEQ ID NO. 1 corresponds to the polynucleotide set forth as SEQ ID NO. 2. SEQ ID NO. 2 is ATGCTGCTCTGCACGGCTCGCCTGGTCGGCCTGCAGCTTCTCATTTCCTGCTGCTGGG CCTTTGCCTGCCATAGCACGGAGTCTTCTCCTGACTTCACCCTCCCCGGAGATTACCT CCTGGCAGGCCTGTTCCCTCTCCATTCTGGCTGTCTGCAGGTGAGGCACAGACCCGA GGTGACCCTGTGTGACAGGTCTTGTAGCTTCAATGAGCATGGCTACCACCTCTTCCA GGCTATGCGGCTTGGGGTTGAGGAGATAAACAACTCCACGGCCCTGCTGCCCAACAT CACCCTGGGGTACCAGCTGTATGATGTGTGTTCTGACTCTGCCAATGTGTATGCCAC GCTGAGAGTGCTCTCCCTGCCAGGGCAACACCACATAGAGCTCCAAGGAGACCTTCT CCACTATTCCCCTACGGTGCTGGCAGTGATTGGGCCTGACAGCACCAACCGTGCTGC CACCACAGCCGCCCTGCTGAGCCCTTTCCTGGTGCCCATGATTAGCTATGCGGCCAG CAGCGAGACGCTCAGCGTGAAGCGGCAGTATCCCTCTTTCCTGCGCACCATCCCCAA TGACAAGTACCAGGTGGAGACCATGGTGCTGCTGCTGCAGAAGTTCGGGTGGACCTG GATCTCTCTGGTTGGCAGCAGTGACGACTATGGGCAGCTAGGGGTGCAGGCACTGGA GAACCAGGCCACTGGTCAGGGGATCTGCATTGCTTTCAAGGACATCATGCCCTTCTC TGCCCAGGTGGGCGATGAGAGGATGCAGTGCCTCATGCGCCACCTGGCCCAGGCCG GGGCCACCGTCGTGGTTGTTTTTTCCAGCCGGCAGTTGGCCAGGGTGTTTTTCGAGTC CGTGGTGCTGACCAACCTGACTGGCAAGGTGTGGGTCGCCTCAGAAGCCTGGGCCCT CTCCAGGCACATCACTGGGGTGCCCGGGATCCAGCGCATTGGGATGGTGCTGGGCGT GGCCATCCAGAAGAGGGCTGTCCCTGGCCTGAAGGCGTTTGAAGAAGCCTATGCCCG GGCAGACAAGAAGGCCCCTAGGCCTTGCCACAAGGGCTCCTGGTGCAGCAGCAATC AGCTCTGCAGAGAATGCCAAGCTTTCATGGCACACACGATGCCCAAGCTCAAAGCCT TCTCCATGAGTTCTGCCTACAACGCATACCGGGCTGTGTATGCGGTGGCCCATGGCC TCCACCAGCTCCTGGGCTGTGCCTCTGGAGCTTGTTCCAGGGGCCGAGTCTACCCCT GGCAGCTTTTGGAGCAGATCCACAAGGTGCATTTCCTTCTACACAAGGACACTGTGG CGTTTAATGACAACAGAGATCCCCTCAGTAGCTATAACATAATTGCCTGGGACTGGA ATGGACCCAAGTGGACCTTCACGGTCCTCGGTTCCTCCACATGGTCTCCAGTTCAGCT AAACATAAATGAGACCAAAATCCAGTGGCACGGAAAGGACAACCAGGTGCCTAAGT CTGTGTGTTCCAGCGACTGTCTTGAAGGGCACCAGCGAGTGGTTACGGGTTTCCATC ACTGCTGCTTTGAGTGTGTGCCCTGTGGGGCTGGGACCTTCCTCAACAAGAGTGACC TCTACAGATGCCAGCCTTGTGGGAAAGAAGAGTGGGCACCTGAGGGAAGCCAGACC TGCTTCCCGCGCACTGTGGTGTTTTTGGCTTTGCGTGAGCACACCTCTTGGGTGCTGC TGGCAGCTAACACGCTGCTGCTGCTGCTGCTGCTTGGGACTGCTGGCCTGTTTGCCTG GCACCTAGACACCCCTGTGGTGAGGTCAGCAGGGGGCCGCCTGTGCTTTCTTATGCT GGGCTCCCTGGCAGCAGGTAGTGGCAGCCTCTATGGCTTCTTTGGGGAACCCACAAG GCCTGCGTGCTTGCTACGCCAGGCCCTCTTTGCCCTTGGTTTCACCATCTTCCTGTCCT GCCTGACAGTTCGCTCATTCCAACTAATCATCATCTTCAAGTTTTCCACCAAGGTACC TACATTCTACCACGCCTGGGTCCAAAACCACGGTGCTGGCCTGTTTGTGATGATCAG CTCAGCGGCCCAGCTGCTTATCTGTCTAACTTGGCTGGTGGTGTGGACCCCACTGCCT GCTAGGGAATACCAGCGCTTCCCCCATCTGGTGATGCTTGAGTGCACAGAGACCAAC TCCCTGGGCTTCATACTGGCCTTCCTCTACAATGGCCTCCTCTCCATCAGTGCCTTTG CCTGCAGCTACCTGGGTAAGGACTTGCCAGAGAACTACAACGAGGCCAAATGTGTC ACCTTCAGCCTGCTCTTCAACTTCGTGTCCTGGATCGCCTTCTTCACCACGGCCAGCG TCTACGACGGCAAGTACCTGCCTGCGGCCAACATGATGGCTGGGCTGAGCAGCCTGA GCAGCGGCTTCGGTGGGTATTTTCTGCCTAAGTGCTACGTGATCCTCTGCCGCCCAG ACCTCAACAGCACAGAGCACTTCCAGGCCTCCATTCAGGACTACACGAGGCGCTGCG GCTCCACCTGA.

In some embodiments, the T1R1 umami taste receptor subunit may correspond to a polynucleotide sequence at least 70% similar to the sequence of SEQ ID NO. 2, at least 80% similar to the sequence of SEQ ID NO. 2, at least 90% similar to the sequence of SEQ ID NO. 2, at least 95% similar to the sequence of SEQ ID NO. 2, at least 96% similar to the sequence of SEQ ID NO. 2, at least 97% similar to the sequence of SEQ ID NO. 2, at least 98% similar to the sequence of SEQ ID NO. 2, at least 99% similar to the sequence of SEQ ID NO. 2, or identical to the sequence of SEQ ID NO. 2.

In some embodiments, a cell, such as a U2OS cell, includes a rat-human T1R3 taste receptor subunit. In certain related aspects, the disclosure provides a cell, such as U2OS cell, that includes an exogenous nucleic acid sequence that encodes for a rat-human T1R3 taste receptor subunit.

In some embodiments, the rat-human T1R3 taste receptor subunit may be a polypeptide set forth as SEQ ID NO. 3, or a functional fragment thereof. SEQ ID NO. 3 is MPGLAILGLSLAAFLELGMGSSLCLSQQFKAQGDYILGGLFPLGTTEEATLNQRTQPN GILCTRFSPLGLFLAMAMKMAVEEINNGSALLPGLRLGYDLFDTCSEPVVTMKPSLMFM AKVGSQSIAAYCNYTQYQPRVLAVIGPHSSELALITGKFFSFFLMPQVSYSASMDRLSDR ETFPSFFRTVPSDRVQLQAVVTLLQNFSWNWVAALGSDDDYGREGLSIFSGLANSRGICI AHEGLVPQHDTSGQQLGKVVDVLRQVNQSKVQVVVLFASARAVYSLFSYSILHDLSPK VWVASESWLTSDLVMTLPNIARVGTVLGFLQRGALLPEFSHYVETRLALAADPTFCASL KAELDLEERVMGPRCSQCDYIMLQNLSSGLMQNLSAGQLHHQIFATYAAVYSVAQALH NTLQCNVSHCHTSEPVQPWQLLENMYNMSFRARDLTLQFDAKGSVDMEYDLKMWVW QSPTPVLHTVGTFNGTLQLQHSKMYWPGNQVPVSQCSRQCKDGQVRRVKGFHSCCYD CVDCKAGSYRKHPDDFTCTPCGKDQWSPEKSTTCLPRRPKFLEWGEPAVLLLLLLLSLA LGLVLAALGLFVHHRDSPLVQASGGPLACFGLVCLGLVCLSVLLFPGQPSPARCLAQQP LSHLPLTGCLSTLFLQAAEIFVESELPLSWADRLSGCLRGPWAWLVVLLAMLVEVALCT WYLVAFPPEVVTDWHMLPTEALVHCRTRSWVSFGLAHATNATLAFLCFLGTFLVRSQP GRYNRARGLTFAMLAYFITWVSFVPLLANVQVVLRPAVQMGALLLCVLGILAAFHLPR CYLLMRQPGLNTPEFFLGGGPGDAQGQNDGNTGNQGKHE.

In some embodiments, the rat-human T1R3 taste receptor subunit may have a polypeptide sequence at least 70% similar to the sequence of SEQ ID NO. 3, at least 80% similar to the sequence of SEQ ID NO. 3, at least 90% similar to the sequence of SEQ ID NO. 3, at least 95% similar to the sequence of SEQ ID NO. 3, at least 96% similar to the sequence of SEQ ID NO. 3, at least 97% similar to the sequence of SEQ ID NO. 3, at least 98% similar to the sequence of SEQ ID NO. 3, at least 99% similar to the sequence of SEQ ID NO. 3, or identical to the sequence of SEQ ID NO. 3.

In some embodiments, the rat-human T1R3 taste receptor subunit polypeptide set forth as SEQ ID NO. 3 corresponds to the polynucleotide set forth as SEQ ID NO. 4. SEQ ID NO. 4 is ATGCCGGGTTTGGCTATCTTGGGCCTCAGTCTGGCTGCTTTCCTGGAGCTTGGGAT GGGGTCCTCTTTGTGTCTGTCACAGCAATTCAAGGCACAAGGGGACTATATATTGGG TGGACTATTTCCCCTGGGCACAACTGAGGAGGCCACTCTCAACCAGAGAACACAGCC CAACGGCATCCTATGTACCAGGTTCTCGCCCCTTGGTTTGTTCCTGGCCATGGCTATG AAGATGGCTGTAGAGGAGATCAACAATGGATCTGCCTTGCTCCCTGGGCTGCGACTG GGCTATGACCTGTTTGACACATGCTCAGAGCCAGTGGTCACCATGAAGCCCAGCCTC ATGTTCATGGCCAAGGTGGGAAGTCAAAGCATTGCTGCCTACTGCAACTACACACAG TACCAACCCCGTGTGCTGGCTGTCATTGGTCCCCACTCATCAGAGCTTGCCCTCATTA CAGGCAAGTTCTTCAGCTTCTTCCTCATGCCACAGGTCAGCTATAGTGCCAGCATGG ATCGGCTAAGTGACCGGGAAACATTTCCATCCTTCTTCCGCACAGTGCCCAGTGACC GGGTGCAGCTGCAGGCCGTTGTGACACTGTTGCAGAATTTCAGCTGGAACTGGGTGG CTGCCTTAGGTAGTGATGATGACTATGGCCGGGAAGGTCTGAGCATCTTTTCTGGTC TGGCCAACTCACGAGGTATCTGCATTGCACACGAGGGCCTGGTGCCACAACATGACA CTAGTGGCCAACAATTGGGCAAGGTGGTGGATGTGCTACGCCAAGTGAACCAAAGC AAAGTACAGGTGGTGGTGCTGTTTGCATCTGCCCGTGCTGTCTACTCCCTTTTTAGCT ACAGCATCCTTCATGACCTCTCACCCAAGGTATGGGTGGCCAGTGAGTCCTGGCTGA CCTCTGACCTGGTCATGACACTTCCCAATATTGCCCGTGTGGGCACTGTTCTTGGGTT TCTGCAGCGCGGTGCCCTACTGCCTGAATTTTCCCATTATGTGGAGACTCGCCTTGCC CTAGCTGCTGACCCAACATTCTGTGCCTCCCTGAAAGCTGAGTTGGATCTGGAGGAG CGCGTGATGGGGCCACGCTGTTCACAATGTGACTACATCATGCTACAGAACCTGTCA TCTGGGCTGATGCAGAACCTATCAGCTGGGCAGTTGCACCACCAAATATTTGCAACC TATGCAGCTGTGTACAGTGTGGCTCAGGCCCTTCACAACACCCTGCAGTGCAATGTC TCACATTGCCACACATCAGAGCCTGTTCAACCCTGGCAGCTCCTGGAGAACATGTAC AATATGAGTTTCCGTGCTCGAGACTTGACACTGCAGTTTGATGCCAAAGGGAGTGTA GACATGGAATATGACCTGAAGATGTGGGTGTGGCAGAGCCCTACACCTGTACTACAT ACTGTAGGCACCTTCAACGGCACCCTTCAGCTGCAGCACTCGAAAATGTATTGGCCA GGCAACCAGGTGCCAGTCTCCCAGTGCTCCCGGCAGTGCAAAGATGGCCAGGTGCG CAGAGTAAAGGGCTTTCATTCCTGCTGCTATGACTGTGTGGACTGCAAGGCAGGGAG CTACCGGAAGCATCCAGATGACTTCACCTGTACTCCATGTGGCAAGGATCAGTGGTC CCCAGAAAAAAGCACAACCTGCTTACCTCGCAGGCCCAAGTTTCTCGAGTGGGGCGA GCCGGCTGTGCTGCTGCTGCTCCTGCTGCTGAGCCTGGCGCTGGGCCTTGTGCTGGCT GCTTTGGGGCTGTTCGTTCACCATCGGGACAGCCCACTGGTTCAGGCCTCGGGGGGG CCCCTGGCCTGCTTTGGCCTGGTGTGCCTGGGCCTGGTCTGCCTCAGCGTCCTCCTGT TCCCTGGCCAGCCCAGCCCTGCCCGATGCCTGGCCCAGCAGCCCTTGTCCCACCTCC CGCTCACGGGCTGCCTGAGCACACTCTTCCTGCAGGCGGCCGAGATCTTCGTGGAGT CAGAACTGCCTCTGAGCTGGGCAGACCGGCTGAGTGGCTGCCTGCGGGGGCCCTGG GCCTGGCTGGTGGTGCTGCTGGCCATGCTGGTGGAGGTCGCACTGTGCACCTGGTAC CTGGTGGCCTTCCCGCCGGAGGTGGTGACGGACTGGCACATGCTGCCCACGGAGGCG CTGGTGCACTGCCGCACACGCTCCTGGGTCAGCTTCGGCCTAGCGCACGCCACCAAT GCCACGCTGGCCTTTCTCTGCTTCCTGGGCACTTTCCTGGTGCGGAGCCAGCCGGGCC GCTACAACCGTGCCCGTGGCCTCACCTTTGCCATGCTGGCCTACTTCATCACCTGGGT CTCCTTTGTGCCCCTCCTGGCCAATGTGCAGGTGGTCCTCAGGCCCGCCGTGCAGAT GGGCGCCCTCCTGCTCTGTGTCCTGGGCATCCTGGCTGCCTTCCACCTGCCCAGGTGT TACCTGCTCATGCGGCAGCCAGGGCTCAACACCCCCGAGTTCTTCCTGGGAGGGGGC CCTGGGGATGCCCAAGGCCAGAATGACGGGAACACAGGAAATCAGGGGAAACATG AGTGA.

In some embodiments, the rat-human T1R3 taste receptor subunit may correspond to a polynucleotide sequence at least 70% similar to the sequence of SEQ ID NO. 4, at least 80% similar to the sequence of SEQ ID NO. 4, at least 90% similar to the sequence of SEQ ID NO. 4, at least 95% similar to the sequence of SEQ ID NO. 4, at least 96% similar to the sequence of SEQ ID NO. 4, at least 97% similar to the sequence of SEQ ID NO. 4, at least 98% similar to the sequence of SEQ ID NO. 4, at least 99% similar to the sequence of SEQ ID NO. 4, or identical to the sequence of SEQ ID NO. 4.

In some embodiments, the G-protein may be a polypeptide set forth as SEQ ID NO. 5, or a functional fragment thereof. SEQ ID NO. 3 is MARSLTWRCCPWCLTEDEKAAARVDQEINRILLEQKKQDRGELKLLLLGPGESGKSTFI KQMRIIHGAGYSEEERKGFRPLVYQNIFVSMRAMIEAMERLQIPFSRPESKHHASLVMSQ DPYKVTTFEKRYAAAMQWLWRDAGIRACYERRREFHLLDSAVYYLSHLERITEEGYVP TAQDVLRSRMPTTGINEYCFSVQKTNLRIVDVGGQKSERKKWIHCFENVIALIYLASLSE YDQCLEENNQENRMKESLALFGTILELPWFKSTSVILFLNKTDILEEKIPTSHLATYFPSFQ GPKQDAEAAKRFILDMYTRMYTGCVDGPEGSKKGARSRRLFSHYTCATDTQNVKFVFD AVTDIIIKENLKDCGLF.

In some embodiments, the G-protein may have a polypeptide sequence at least 70% similar to the sequence of SEQ ID NO. 5, at least 80% similar to the sequence of SEQ ID NO. 5, at least 90% similar to the sequence of SEQ ID NO. 5, at least 95% similar to the sequence of SEQ ID NO. 5, at least 96% similar to the sequence of SEQ ID NO. 5, at least 97% similar to the sequence of SEQ ID NO. 5, at least 98% similar to the sequence of SEQ ID NO. 5, at least 99% similar to the sequence of SEQ ID NO. 5, or identical to the sequence of SEQ ID NO. 5.

In some embodiments, the G-protein set forth as SEQ ID NO. 5 corresponds to the polynucleotide set forth as SEQ ID NO. 6. SEQ ID NO. 6 is ATGGCCCGCTCGCTGACCTGGCGCTGCTGCCCCTGGTGCCTGACGGAGGATGAGA AGGCCGCCGCCCGGGTGGACCAGGAGATCAACAGGATCCTCTTGGAGCAGAAGAAG CAGGACCGCGGGGAGCTGAAGCTGCTGCTTTTGGGCCCAGGCGAGAGCGGGAAGAG CACCTTCATCAAGCAGATGCGGATCATCCACGGCGCCGGCTACTCGGAGGAGGAGC GCAAGGGCTTCCGGCCCCTGGTCTACCAGAACATCTTCGTGTCCATGCGGGCCATGA TCGAGGCCATGGAGCGGCTGCAGATTCCATTCAGCAGGCCCGAGAGCAAGCACCAC GCTAGCCTGGTCATGAGCCAGGACCCCTATAAAGTGACCACGTTTGAGAAGCGCTAC GCTGCGGCCATGCAGTGGCTGTGGAGGGATGCCGGCATCCGGGCCTGCTATGAGCGT CGGCGGGAATTCCACCTGCTCGATTCAGCCGTGTACTACCTGTCCCACCTGGAGCGC ATCACCGAGGAGGGCTACGTCCCCACAGCTCAGGACGTGCTCCGCAGCCGCATGCCC ACCACTGGCATCAACGAGTACTGCTTCTCCGTGCAGAAAACCAACCTGCGGATCGTG GACGTCGGGGGCCAGAAGTCAGAGCGTAAGAAATGGATCCATTGTTTCGAGAACGT GATCGCCCTCATCTACCTGGCCTCACTGAGTGAATACGACCAGTGCCTGGAGGAGAA CAACCAGGAGAACCGCATGAAGGAGAGCCTCGCATTGTTTGGGACTATCCTGGAACT ACCCTGGTTCAAAAGCACATCCGTCATCCTCTTTCTCAACAAAACCGACATCCTGGA GGAGAAAATCCCCACCTCCCACCTGGCTACCTATTTCCCCAGTTTCCAGGGCCCTAA GCAGGATGCTGAGGCAGCCAAGAGGTTCATCCTGGACATGTACACGAGGATGTACA CCGGGTGCGTGGACGGCCCCGAGGGCAGCAAGAAGGGCGCACGATCCCGACGCCTT TTCAGCCACTACACATGTGCCACAGACACACAAAACGTCAAATTCGTGTTTGATGCC GTGACAGATATAATAATAAAAGAGAACCTCAAAGACTGTGGGCTCTTCTGA.

In some embodiments, the rat-human T1R3 taste receptor subunit may correspond to a polynucleotide sequence at least 70% similar to the sequence of SEQ ID NO. 6, at least 80% similar to the sequence of SEQ ID NO. 6, at least 90% similar to the sequence of SEQ ID NO. 6, at least 95% similar to the sequence of SEQ ID NO. 6, at least 96% similar to the sequence of SEQ ID NO. 6, at least 97% similar to the sequence of SEQ ID NO. 6, at least 98% similar to the sequence of SEQ ID NO. 6, at least 99% similar to the sequence of SEQ ID NO. 6, or identical to the sequence of SEQ ID NO. 6.

In some embodiments, a cell, such as a U2OS cell, may be engineered to overexpress one or more T1R1 umami taste receptor proteins. The cell can be engineered to overexpress any of the T1R1 umami taste receptors, such as the human umami taste receptors. In some embodiments, the U2OS cells are engineered to overexpress one or more of the polypeptides set forth as SEQ ID NO. 1.

In some embodiments, a cell, such as a U2OS cell, may be engineered to overexpress one or more T1R3 taste receptor proteins. The cell can be engineered to overexpress a chimeric rat-human T1R3 taste receptors. In some embodiments, the U2OS cells are engineered to overexpress one or more of the polypeptides set forth as SEQ ID NO. 3.

In some embodiments, a cell, such as a U2OS cell, includes an exogenous nucleic acid sequence that encodes for a G-protein. In some further embodiments, the G-protein may be gustducin, such as an α-gustducin. In some embodiments, the G-protein may be a promiscuous G-protein. Non-limiting examples of promiscuous G-proteins include Ga16, Ga15, G15, Ga16gust25, G16gust25, Ga15gust25, Ga16gust44, Ga15gust44, and Ga15-i/3-5, which are described in U.S. Pat. No. 10,107,794. In some embodiments, the G-protein may be a G(i/o) protein. In some embodiments, the G-protein may be G15, G16, G16T44, or G16gust25.

In some embodiments, the cell, such as a U2OS cell, includes a G-protein. In some further embodiments, the G-protein may be gustducin, such as an α-gustducin. In some other examples, the G-protein may be a promiscuous G-protein. Non-limiting examples of promiscuous G-proteins include Ga16, Ga15, Ga16gust25, Ga15gust25, Ga16gust44, Ga15gust44, and Ga15-i/3-5, which are described in U.S. Pat. No. 10,107,794. In some other embodiments, the G-protein may be a G(i/o) protein.

Human U2OS cells are well known in the art and are generally available commercially, for example, from ATCC Catalog No. HTB-96. In some embodiments, the U2OS cells are isolated U2OS cells. In some embodiments, the U2OS cells are contained in an assay. In some embodiments, the U2OS cells are adhered to a substrate. For example, the U2OS cells may be adhered to a surface within a well of a multi-well plate.

In some embodiments, cells other than human U2OS cells may be used. Non-limiting examples of such cells include the following cell types: 1A2, ARH-77, RWPE-1, WI-38, EJM, NCI-H1155, L-1236, NCI-H526, JM1, SHP-77, SNU-878, NCI-H2196, C3A, CA46, SNU-466, KS-1, SNU-738, MOLP-2, HDLM-2, Pfeiffer, HCC-15, Alexander cells, L-540, KMS-12-BM, JK-1, NCI-H1092, SW 1990, NCI-H1184, SU-DHL-1, Hep 3B2.1-7, P3HR-1, NCI-H2029, SU-DHL-5, SNU-1, MOLP-8, SUP-M2, MONO-MAC-1, SNU-1040, KYM-1, HEC-59, HCC1569, OCI-LY3, Hs 819.T, DU4475, CI-1, S-117, OVCAR-8, SNU-626, HL-60, SUIT-2, T3M-4, RKO, MOR/CPR, DK-MG, GA-10, OCUM-1, HCT-15, HT, MONO-MAC-6, G-402, Toledo, COV362, SU-DHL-8, Daoy, NCI-H1435, LS513, Hs 839.T, Hs 172.T, BT-483, KMS-21BM, AGS, NCI-H2172, LC-1/sq-SF, SNU-201, NUGC-4, SK-HEP-1, SUP-B115, SNU-5, HT-1197, SUP-T1, AMO-1, KU812, AN3 CA, AML-193, VMRC-RCW, HLE, HuH28, Hs 751.T, NCI-H2110, MEG-01, MV-4-11, Hep G2, KYSE-30, KALS-1, BICR 6, RMUG-S, JHH-6, Ki-JK, IST-MES1, HCC-95, HPB-ALL, HSC-3, 697, LOU-NH91, KARPAS-299, GI-1, COLO 792, SK-N-FI, D341 Med, HGC-27, SR-786, COLO-818, MHH-CALL-2, SF126, NCI-H322, A-253, NCI-H1623, MCF7, HCC-44, FU97, OCI-LY-19, Hs 766T, NCI-H522, RL, HCC1428, RPMI 6666, U-937, NCI-H460, SW 1088, NCI-H1792, NCI-H1693, UACC-257, JHUEM-2, HuT 78, UACC-893, NCI-H929, A-704, OV56, LN-229, OE19, SK-MEL-24, RD-ES, NCI-H211, KCI-MOH1, NCI-H1963, Hs 706.T, ChaGo-K-1, EPLC-272H, OPM-2, KHM-1B, A549, HuG1-N, NCI-H508, MHH-CALL-3, SNU-1076, A3/KAW, MEL-HO, TO 175.T, Caki-1, Hs 936.T, SK-LU-1, WM-983B, K-562, EFE-184, SNU-520, NCI-H2291, HCC-1195, ABC-1, KE-39, NH-6, HCC2218, CMK, RS4; 11, KYSE-450, OV7, KYSE-510, SK-UT-1, SNU-C1, OE33, P12-ICHIKAWA, DLD-1, COV434, HuNS1, SNU-899, SW480, COLO-678, LU99, KOPN-8, NCI-H2227, SW1463, Hs 675.T, JHH-4, NCI-H1703, HEC-1-A, BDCM, MIA PaCa-2, PC-3, TE-15, PK-45H, MKN-45, HCC-366, CAL-29, HEC-50B, CPC-N, KMRC-20, SW1116, EOL-1, COLO 205, EHEB, YD-38, MC116, SK-N-BE(2), BV-173, NCI-H2347, LU65, RT4, U-87 MG, LK-2, KP-N-YN, HEC-251, NCI-H1651, GP2d, RERF-LC-MS, NB-4, NCI-H2286, SNU-61, T-47D, huH-1, KYSE-180, ST486, SW 1353, M-07e, KASUMI-1, YH-13, NCI-H28, GAMG, JeKo-1, GOS-3, SNU-324, PA-TU-8902, MFE-280, SNU-245, NALM-1, RERF-LC-Sq1, BICR 22, ZR-75-1, COR-L23, SW579, COR-L88, KM12, Hs 611.T, OUMS-23, RERF-LC-Ad1, NCI-H1385, SK-LMS-1, COLO-320, BL-70, GRANTA-519, MCAS, Panc 08.13, AM-38, KMS-11, SIG-M5, SNU-407, JHOS-2, OVCAR-4, Set-2, OV-90, MeWo, HEL, HT-29, MDA-MB-231, TOV-21G, NCI-H1355, KMS-27, NALM-6, KMS-26, Caov-4, KASUMI-2, UACC-62, U266B1, Hs 695T, HT55, BICR 31, TCC-PAN2, KMS-20, Hs 578T, RI-1, Hs 606.T, NCI-H1341, THP-1, BCP-1, Hs 737.T, SW1417, MOLT-4, Raji, ESS-1, MEL-JUSO, SH-10-TC, Hs 683, ME-1, EB2, PLC/PRF/5, NCI-H1339, A4/Fuk, SEM, HEC-265, IST-MES2, KE-97, NCI-H1437, COLO-704, NCI-H1915, TE-5, NCI-H2023, NCI-H82, T1-73, SNU-840, HuT 102, NCI-H1944, KYSE-520, Kasumi-6, 1321N1, Hs 742.T, IM95, PL45, CL-40, WM1799, KMM-1, SNU-449, JHUEM-1, KARPAS-620, Loucy, SNU-1079, Daudi, HCC-56, HSC-2, COR-L47, PA-TU-89885, OAW28, COR-L311, L-363, Malme-3M, NOMO-1, Hs 870.T, SU-DHL-10, Hs 229.T, NCI-H810, KYSE-410, RPMI-8402, SNU-175, EBC-1, RVH-421, K029AX, PA-TU-8988T, LXF-289, OVSAHO, CAL-12T, Hs 940.T, MM1-S, SUP-HD1, LNCaP clone FGC, HSC-4, NU-DHL-1, NCI-H2228, BEN, CAL-78, Sq-1, NCI-H1793, SNU-C2A, MDA-MB-134-VI, COV318, KE-37, TYK-nu, MOTN-1, T98G, SW837, EB1, Becker, PE/CA-PJ34 (clone C12), Hs 616.T, NCI-H446, WM-88, CHP-126, Calu-1, SNU-283, NCI-H1573, SW 1271, SNU-16, JHOS-4, ACHN, Calu-3, KMRC-1, SW 1783, TE-11, TE-9, HuH-6, P31/FUJ, HT-1376, NCI-H520, 786-0, KNS-60, Caki-2, OVK18, PL-21, NCI-H2452, JURL-MK1, TEN, JHH-7, MDA-MB-157, Calu-6, RKN, NUGC-2, ONS-76, J82, OUMS-27, SNU-1196, Hs 739.T, RPMI-7951, NCI-H854, JHH-5, JVM-2, Hey-A8, 5637, KYSE-140, Capan-2, KYSE-150, HEC-1-B, BICR 16, HEL 92.1.7, MHH-NB-11, SNU-387, SK-OV-3, SK-MEL-28, IGROV1, ML-1, HLF-a, CHL-1, YKG1, A-204, OCI-M1, 8505C, JVM-3, NCI-H647, DB, COLO-800, PK-59, FaDu, HLF, OVMANA, EFO-27, PF-382, NCI-H747, LS123, SU-DHL-6, SJRH30, PANC-1, NCI-H2342, KM-H2, DND-41, HH, HuCCT1, F-36P, DMS 454, Hs 274.T, AU565, NCI-H1666, EN, RH-41, NCI-H1373, NCI-H838, SK-MEL-30, MOLM-6, DEL, NCI-H226, NCI-H1648, NCI-H661, 143B, Mino, C32, KMS-34, NCI-H1694, SK-ES-1, UACC-812, GDM-1, NCI-H23, Panc 02.03, CCF-STTG1, LOX IMVI, SJSA-1, MDST8, PK-1, NCI-H716, SU-DHL-4, MPP 89, MJ, COLO 829, PE/CA-PJ15, HD-MY-Z, BxPC-3, WM-793, COLO 668, T84, JHOM-1, PEER, LS411N, GMS-10, KMBC-2, RMG-I, KELLY, SNU-761, NALM-19, HEC-151, G-361, OVTOKO, A-498, SW 900, LCLC-103H, FTC-133, QGP-1, Reh, CMK-11-5, NU-DUL-1, BT-20, Hs 600.T, Hs 604.T, KATO III, SNU-410, NCI-H2126, SK-MEL-5, MDA-MB-468, AsPC-1, HUP-T3, KP-N-SI9s, L-428, SNU-1105, HUP-T4, 769-P, LMSU, NCI-H1869, NCO2, MOLM-16, CAL 27, HCC70, NCI-H1930, COV644, Hs 863.T, HCC-2279, D283 Med, Hs 944.T, HCC1599, MDA-MB-415, HCC2157, NCI-H1618, SNU-308, HCC1954, DMS 153, HPAF-II, T24, CJM, VM-CUB1, UM-UC-3, LAMA-84, NCI-H1734, JHH-2, VMRC-RCZ, MFE-319, MDA-MB-453, SNU-503, TOV-112D, B-CPAP, GSU, HCC-78, NCI-H2171, CAMA-1, HEC-108, HCC4006, CAL-85-1, NCI-H2122, COLO-699, NCI-H196, LUDLU-1, SW 780, RPMI 8226, LP-1, PC-14, HuTu 80, T.T, SW948, 22Rv1, HARA, NCI-H596, IPC-298, SCaBER, NCI-H1838, NB-1, Hs 934.T, Hs 895.T, DMS 114, KYSE-70, KP-3, KP4, DAN-G, NCI-H2009, OC 316, SCC-25, U-138 MG, RCC10RGB, MFE-296, NCI-H1755, RERF-LC-KJ, 8305C, WSU-DLCL2, ES-2, MSTO-211H, SCC-15, ZR-75-30, PSN1, SNU-423, NCI-H2106, TE-1, UT-7, KMS-28BM, NCI-H2081, SK-MM-2, COLO 741, OC 314, HCC1395, MOLT-13, LN-18, Pane 10.05, PE/CA-PJ41 (clone D2), Hs 746T, CW-2, SKM-1, NUGC-3, TE-10, NCI-H358, NCI-H69, BFTC-909, HOS, BICR 18, NCI-H1395, OVKATE, Hs 698.T, EFM-19, COLO-783, MHH-CALL-4, ACC-MESO-1, NCI-H1436, KP-N-RT-BM-1, SK-MEL-31, NCI-H1105, CAL-51, YD-15, NCI-H2085, NCI-H2444, HCC1187, Hs 939.T, CAL-120, SCC-9, TUHR14TKB, KMRC-2, KG-1-C, ECC10, CGTH-W-1, NCI-H841, C2BBe1, SUP-T1l, RCH-ACV, CADO-ES1, JURKAT, 647-V, SK-MEL-2, MDA-MB-175-VII, MKN74, SNU-C4, LCLC-97TM1, SCC-4, BHY, IGR-37, KYO-1, Hs 281.T, TT, TUHR4TKB, HT-1080, NCI-H660, TE 441.T, LS1034, KNS-42, Pane 04.03, HCC1419, AZ-521, SNG-M, NCI-N87, G-292, clone A141B1, KPL-1, MDA-MB-361, CL-14, NCI-H2170, HuH-7, RD, NCI-H2066, IGR-1, TE-14, VCaP, BL-41, SNU-620, SK-MES-1, MEC-2, NCI-H1299, IGR-39, RT112/84, SF-295, DV-90, A2780, BICR 56, NCI-H510, NCI-H2141, YD-8, NCI-H2405, TF-1, MEC-1, CCK-81, NCI-H1048, Hs 822.T, NCI-H2052, K052, CAL-54, Hs 840.T, SW620, SK-CO-1, BT-474, CL-11, KNS-62, NCI-H1650, G-401, MOLT-16, SNU-398, COLO-680N, EM-2, Hs 294T, CAL-62, KMRC-3, A101D, KG-1, BT-549, HT115, A-375, SW-1710, WM-115, KLE, JHUEM-3, MKN7, CHP-212, HCC202, BC-3C, NCI-H1568, KMS-18, PE/CA-PJ49, COLO-849, SIMA, OCI-AML3, GSS, EC-GI-10, EFO-21, RCM-1, DMS 273, KU-19-19, RERF-GC-1B, SH-4, SK-MEL-3, RERF-LC-Ad2, M059K, JHOM-2B, MDA PCa 2b, Hs 852.T, RL95-2, Pane 03.27, SNU-216, Pane 02.13, CFPAC-1, SK-N-SH, OCI-AML2, LoVo, SBC-5, NCI-H1876, NCI-H441, SK-N-AS, COR-L24, HCC38, NCI-H1781, DOHH-2, NCI-H1563, U-251 MG, HPAC, JIMT-1, U-2 OS, A-673, TC-71, NCI-H650, NIH:OVCAR-3, CAS-1, JL-1, SK-MEL-1, MDA-MB-4355, Ishikawa (Heraklio) 02 ER-, TE 617.T, SU.86.86, RERF-LC-AI, TT2609-C02, LS 180, YAPC, HDQ-P1, KNS-81, FU-OV-1, KP-2, DMS 53, SNU-1272, Detroit 562, 42-MG-BA, L3.3, COLO-679, NCI-H2087, NCI-H2030, GCT, NCI-H889, Caov-3, MDA-MB-436, NCI-H524, MKN1, KCL-22, Capan-1, CML-T1, H4, NCI-H727, Hs 343.T, MHH-ES-1, NMC-G1, HCC-1171, REC-1, Hs 618.T, A172, YD-10B, SW48, MUTZ-5, TE-6, JHH-1, HCT 116, TE-4, IA-LM, MG-63, NCI-H1975, TALL-1, HCC1806, HMCB, SCLC-21H, HCC1500, CL-34, Pane 05.04, SW403, TM-31, HCC1937, JMSU-1, DMS 79, SNB-19, NCI-H1836, Li-7, HCC827, 639-V, MOLM-13, SK-BR-3, IMR-32, TUHR10TKB, OAW42, SK-N-MC, TGBC11TKB, NCI-H1581, EFM-192A, YMB-1, HCC2935, ECC12, HCC-33, DU 145, NCI-H146, SNU-1214, SNU-1077, 23132/87, HT-144, SNU-182, Hs 888.T, SNU-475, GCIY, Hs 729, JHOC-5, SW 1573, HEC-6, OCI-AMLS, Hs 688(A).T, Hs 821.T, PCM6, RT-112, SK-N-DZ, SNU-478, SNU-119, HCC1143, NCI-H209, 8-MG-BA, COR-L105, COR-L95, SNU-46, COV504, CAL-148, SNU-05, DBTRG-05MG, BHT-101, WM-266-4, BFTC-905, KYSE-270, TE-8, SNU-213, and SH-SYSY.

### Screening Methods

The combinations of cells and receptors discussed above may be used within cell-based methods to screen compounds. For example, the cells and receptors may be used to identify compounds having some effect on umami taste sensing. Various functional assays can be used, including assays that measure of changes in intracellular calcium cation concentration (for example, Fluorometric Imaging Plate Reader-based Ca²⁺ mobilization assay, FLIPR), pERK1/2 activation (for example, high content imaging, HCl), and receptor internalization (for example, TRANSFLUOR). A change in T1R1 umami taste receptor activity by a test compound or by an umami tastant in the presence of a test compound indicates modulation of the T1R1 umami taste receptor by the test compound, thereby identifying it as a T1R1 umami taste modulator, namely, a molecule that provides an umami taste, a molecule that blocks some or all of an umami taste of an umami tastant, or a molecule that enhances the umami taste of another molecule. A change in T1R1 umami taste receptor activity may indicate that the test compound is an umami taste modulator.

Some embodiments include a method of identifying an umami tastant, the method comprising: (a) contacting one or more U2OS cells expressing the umami taste receptors (according to any of the examples set forth above) with a test compound; and (b) measuring the T1R1+3 umami taste receptor activity, wherein a change in the T1R1+3 umami taste activity may indicate that the test compound is an umami tastant. In some examples, the method includes identifying an active test compound that elicits umami taste based on the measured response. In some examples, the method includes selecting the active test compound as a compound that elicits umami taste. An increase in T1R1+3 taste receptor activity indicates that the compound may elicit an umami taste. Activation of a T1R1+3 taste receptor may be achieved when the receptor activity value relative to the control is 110%, optionally 150%, 200-500%, or 1000-2000%. Inhibition of a T1R1+3 taste receptor may be achieved when the receptor activity value relative to a control is no more than 90%, no more than 80%, no more than 70%, no more than 60%, no more than 50%, no more than 30%, or no more than 30%.

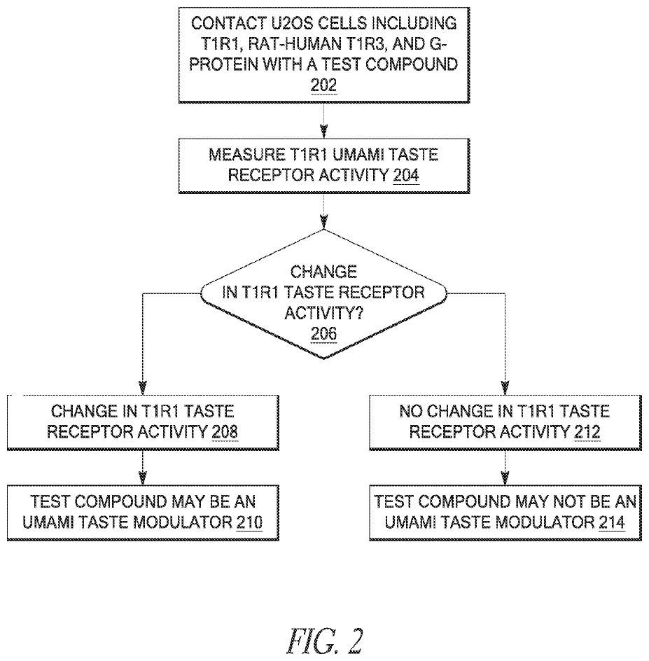

FIG. 2 schematically illustrates an example method for determining whether a test compound may be an umami tastant. The method begins by contacting U2OS cells including a T1R1 umami taste receptor subunit, a rat-human T1R3 taste receptor subunit, and a G-protein with a test compound, as shown in 202. Next, the method proceeds by measuring T1R1+3 umami taste receptor activity, as in 204. In some examples, G-protein increases intracellular Ca²⁺ in response to T1R1+3 sensing an umami compound. Thus, a method known in the art for detecting intracellular Ca²⁺ may measure T1R1+3 response. Using such a method, a change in T1R1+3 taste receptor activity may be determined, 206. If there is a change in T1R1+3 taste receptor activity 208, the method indicates that the test compound is an umami tastant, 210. If there is no change in T1R1+3 taste receptor activity 212, the method indicates the test compound may not be an umami tastant 214.

In other embodiments, the effect of a test compound on umami taste may be evaluated in the presence of a known umami tastant. Thus, some embodiments provide a method of identifying compounds that enhance or reduce umami taste elicited by the known umami tastant, the method includes: (a) contacting one or more U2OS cells expressing the umami taste receptors of the first aspect with a test compound and an umami tastant; (b) measuring a response of the T1R1 umami taste receptor by comparing an activity of the T1R1 umami taste receptor to the umami tastant in the presence and absence of the test compound, wherein a change in the T1R1+3 umami taste receptor activity may indicate that the test compound is an umami taste modulator. In some examples, the method includes identifying a test compound as an umami taste modulator based on the change in the T1R1 umami taste receptor activity. In some examples, the method includes selecting the test compound as a compound that enhances or reduces umami taste as demonstrated by the enhancement or inhibition of T1R1+3 activity in the assay. An enhancement in T1R1+3 taste receptor activity indicates that the compound may enhance umami taste. Enhancement of a T1R1+3 taste receptor may be achieved when the receptor activity value relative to the activity of the umami tastant control is 110%, optionally 150%, 200-500%, or 1000-2000%. A decrease in T1R1+3 taste receptor activity indicates that the compound may block (or decrease) umami taste. Inhibition of T1R1+3 receptor may be achieved when the receptor activity value relative to the activity of the umami tastant control is about 90%, optionally 50%, or optionally 25-0%.

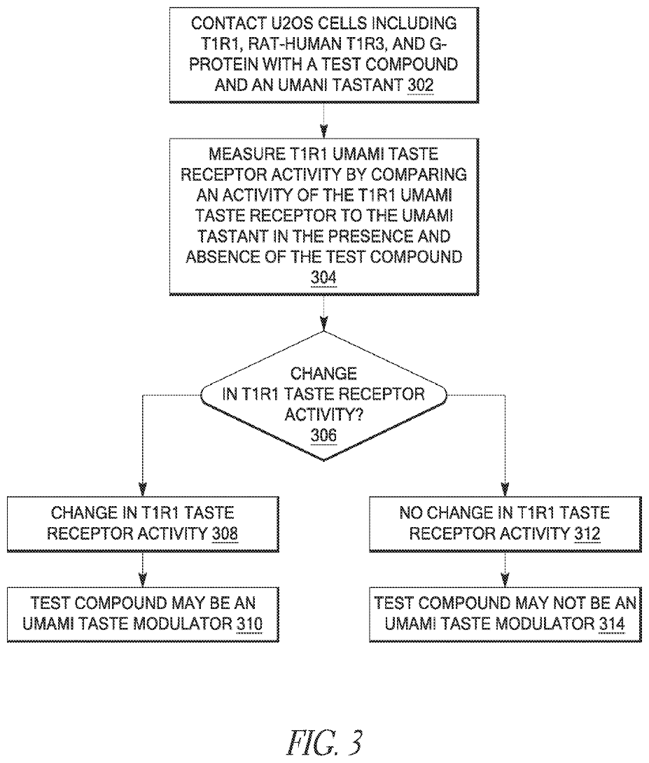

FIG. 3 schematically illustrates an example method for determining whether a test compound modulates umami taste in the presence of a known umami tastant. The method begins by contacting U2OS cells expressing the T1R1 subunit, the rat-human T1R3 subunit, and a G-protein with a test compound and an umami tastant, 302. Next, the method proceeds by measuring the umami taste receptor activity by comparing an activity of the umami taste receptor to the umami tastant in the presence and absence of the test compound, as provided in 304. Using such a method, a change in taste receptor activity may be determined, 306. If there is a change in umami taste receptor activity, 308, the method indicates that the test compound may be an umami taste modulator, 310. If there is no change in umami taste receptor activity, 312, the method indicates the test compound is not an umami taste modulator, 314.

Contacting the cells with the test compound described above can be carried out in any suitable way. For example, in some non-limiting instances, when contacting the test compound (and, in certain aspects, an umami tastant) to a cell that includes a T1R1+3 umami taste receptor, the contact can be carried out in a cell-based assay. Such cell-based assays are well known in the art.

For example, G-proteins coupled to a T1R1+3 umami taste receptor may increase intracellular Ca²⁺ when an umami compound is sensed. A Ca²⁺-sensitive fluorescent dye, for example, may optically indicate such an increase in intracellular Ca²⁺. Pairing the Ca²⁺-sensitive fluorescent dye with an imaging method may allow for measurement of T1R1+3 activity, as described in the art. As an example, Fluo-4 may be used as a dye for fluorescent detection of changes in intracellular Ca²⁺ concentration.

The foregoing examples involve measuring the response of a taste receptor protein to the test compound. This measuring can be carried out by any suitable means. For example, in some examples, each taste receptor protein may be expressed on the surface of a cell, and compositions that include the test compound are screened against the cells expressing a taste receptor protein in a standard cellular assay. Measuring the binding can be carried out by any suitable means typically used in determining protein binding in cellular assays. Suitable methods include, but are not limited to, use of calcium fluorescent dyes, a calcium indicator protein, a fluorescent cAMP indicator, and the like. Activity of the test compound may be determined by its ability to antagonize binding of the umami taste compound to one or more of the taste receptor proteins. In some examples, the U2OS cells include a detectable label or an exogenous nucleic acid sequence that encodes a detectable label. In some examples, the detectable label may be (3-arrestin GFP.

Changes in ion flux may be assessed by determining changes in ionic polarization (i.e., electrical potential) of the cell or membrane expressing a T1R1+3 receptor. One means to determine changes in cellular polarization is by measuring changes in current (thereby measuring changes in polarization) with voltage-clamp and patch-clamp techniques (see, e.g., the “cell-attached” mode, the “inside-out” mode, and the “whole cell” mode, e.g., Ackerman et al., New Engl. J Med., 336:1575-1595 (1997)). Whole cell currents are conveniently determined using the standard. Other known assays may include: radiolabeled ion flux assays and fluorescence assays using voltage-sensitive dyes (see, e.g., Vestergarrd-Bogind et al., J. Membrane Biol., 88:67-75 (1988); Gonzales & Tsien, Chem. Biol., 4:269277 (1997); Daniel et al., J. Pharmacol. Meth., 25:185-193 (1991); Holevinsky et al., J. Membrane Biology, 137:59-70 (1994)).

The effects of the test compounds upon the function of the T1R1+3 receptor can be measured by examining any of the parameters described above. Any suitable physiological change that affects GPCR activity can be used to assess the influence of a test compound on the receptor of this example. When the functional consequences are determined using intact cells or animals, one can also measure a variety of effects such as transmitter release, hormone release, transcriptional changes to both known and uncharacterized genetic markers (e.g., northern blots), changes in cell metabolism such as cell growth or pH changes, and changes in intracellular second messengers such as Ca²⁺, IP3, cGMP, or cAMP.

Some example assays for GPCRs include cells that are loaded with ion or voltage sensitive dyes to report receptor activity. Assays for determining activity of such receptors can also use known agonists and antagonists for other G protein-coupled receptors as controls to assess activity of tested compounds. In assays for identifying modulatory compounds (e.g., agonists, antagonists), changes in the level of ions in the cytoplasm or membrane voltage will be monitored using an ion sensitive or membrane voltage fluorescent indicator, respectively. Among the ion-sensitive indicators and voltage probes that may be employed are those disclosed in the Molecular Probes 1997 Catalog. For G protein-coupled receptors, promiscuous G proteins such as Ga15 and Ga16 can be used in the assay of choice (Wilkie et al., Proc. Nat'l Acad. Sci., 88:10049-10053 (1991)).

Receptor activation initiates subsequent intracellular events, e.g., increases in second messengers. Activation of some G protein-coupled receptors stimulates the formation of inositol triphosphate (IP3) through phospholipase C-mediated hydrolysis of phosphatidylinositol (Berridge & Irvine, Nature, 312:315-21 (1984)). IP3 in turn stimulates the release of intracellular calcium ion stores. Thus, a change in cytoplasmic calcium ion levels, or a change in second messenger levels such as IP3 can be used to assess G protein-coupled receptor function. Cells expressing such G protein-coupled receptors may exhibit increased cytoplasmic calcium levels as a result of contribution from both calcium release from intracellular stores and extracellular calcium entry via plasma membrane ion channels.

In an example, T1R1+3 receptor activity is measured by stably or transiently co-expressing T1R1 and rat-human T1R3 genes, in a heterologous cell with a promiscuous G protein that links the receptor to a phospholipase C signal transduction pathway (see Offermanns & Simon, J. Biol. Chem., 270:15175-15180 (1995)). In an example, the cell line is U2OS (which does not normally express T1R1+3 genes) and the promiscuous G protein is Ga15 (Offermanns & Simon, supra). Modulation of taste transduction is assayed by measuring changes in intracellular Ca²⁺ levels, which may change in response to modulation of the T1R1+3 receptor signal transduction pathway via administration of a molecule that associates with and activates the receptor. Changes in Ca²⁺ levels are optionally measured using fluorescent Ca²⁺ indicator dyes and fluorometric imaging.

In another embodiment, phosphatidyl inositol (PI) hydrolysis can be analyzed according to U.S. Pat. No. 5,436,128, herein incorporated by reference. Briefly, the assay involves labeling of cells with 3H-myoinositol for 48 or more hrs. The labeled cells are treated with a test compound for one hour. The treated cells are lysed and extracted in chloroform-methanol-water after which the inositol phosphates were separated by ion exchange chromatography and quantified by scintillation counting. Fold stimulation is determined by calculating the ratio of cpm in the presence of agonist, to cpm in the presence of buffer control. Likewise, fold inhibition is determined by calculating the ratio of cpm in the presence of antagonist, to cpm in the presence of buffer control (which may or may not contain an agonist).

Other receptor assays can involve determining the level of intracellular cyclic nucleotides, e.g., cAMP or cGMP. In cases where activation of the receptor results in a decrease in cyclic nucleotide levels, it may be preferable to expose the cells to agents that increase intracellular cyclic nucleotide levels, e.g., forskolin, prior to adding a receptor-activating compound to the cells in the assay. In one embodiment, the changes in intracellular cAMP or cGMP can be measured using immunoassays. The method described in Offermanns & Simon, J. Bio. Chem., 270:15175-15180 (1995), maybe used to determine the level of cAMP. Also, the method described in Pelley-Bosco et al., Am. J. Resp. Cell and Mal. Biol., 11:159-164 (1994), maybe used to determine the level of cGMP. Further, an assay kit for measuring cAMP and/or cGMP is described in U.S. Pat. No. 4,115,538, herein incorporated by reference. In another embodiment, transcription levels can be measured to assess the effects of a test compound on signal transduction. A host cell expressing the T1R1+3 receptor is contacted with a test compound for a sufficient time to effect any interactions, and then the level of gene expression is measured. The amount of time to effect such interactions may be empirically determined, such as by running a time course and measuring the level of transcription as a function of time. The amount of transcription may be measured by using any method known to those of skill in the art to be suitable. For example, mRNA expression of the protein of interest may be detected using northern blots or their polypeptide products may be identified using immunoassays. Alternatively, transcription-based assays using reporter gene may be used as described in U.S. Pat. No. 5,436,128, herein incorporated by reference. The reporter genes can be, e.g., chloramphenicol acetyltransferase, luciferase, beta-galactosidase beta-lactamase and alkaline phosphatase. Furthermore, the protein of interest can be used as an indirect reporter via attachment to a second reporter such as green fluorescent protein (see, e.g., Mistili & Spector, Nature Biotechnology, 15:961-964 (1997)).

The amount of transcription is then compared to the amount of transcription in either the same cell in the absence of the test compound, or it may be compared with the amount of transcription in a substantially identical cell that lacks the T1R1+3 receptor. A substantially identical cell may be derived from the same cells from which the recombinant cell was prepared but which had not been modified by introduction of heterologous DNA. Any difference in the amount of transcription indicates that the test compound has in some manner altered the activity of the T1R1+3 receptor.

In some further examples, the methods further include selecting the active test compound as a compound that reduces umami taste. The selecting can be carried out by any suitable means once the active test compounds are identified.

The test compound can be any suitable compound that may be amenable for use in cellular screening assays. In some examples, the test compound may be an organic compound. In some examples, the test compound may be a naturally occurring compound or a glycosylated derivative thereof. In some other examples, the test compound may be a non-naturally occurring compound.

In aspects and examples that employ an umami tastant, the umami tastant can be any suitable compound that functions as an umami tastant, namely, any compound that agonizes one or more T1R1 umami taste receptors. Non-limiting examples of umami tastants suitable for use in such assays include monosodium glutamate, L-aspartate, L-glutamate, L-inosinate, L-guanylate, purine 50-ribonucleotides including inosine 50-monophosphate and guanosine 50-monophosphate, and active pharmaceutical ingredients.

### Uses, Methods, and Ingestible Compositions

In some examples, compounds identified by the cell-based assays discussed above may be incorporated into ingestible compositions, used in food preparation, or incorporated into methods of preparing food. In other aspects, the disclosure provides uses of any identified or selected active compounds of the foregoing aspects, including any examples or combination of examples thereof, as set forth above.

In a certain aspect, the disclosure provides uses of any identified or selected active test compounds of the screening methods disclosed herein to provide or enhance umami taste to an ingestible composition. In related aspects, the disclosure provides methods of enhancing an umami taste of an ingestible composition, the method includes introducing an amount (such as an umami-reducing effective amount) of any identified or selected active test compounds of the screening methods disclosed herein to an ingestible composition.

The foregoing uses and methods generally involve the use of the identified or selected active compounds in a composition containing one or more additional ingredients. For example, in at least one aspect, the disclosure provides compositions includes any identified or selected active compounds of the foregoing aspects, including any examples or combination of examples thereof, as set forth above, wherein the identified or selected active compounds make up at least 50% by weight of the compositions on a dry weight basis (e.g., based on the total weight of the composition excluding the weight of any liquid carrier). In a related aspect, the disclosure provides solid-state compositions includes any identified or selected active compounds of the foregoing aspects, including any examples or combination of examples thereof, as set forth above, wherein the identified or selected active compounds make up at least 50% by weight of the solid-state compositions, based on the total weight of composition. In another related aspect, the disclosure provides ingestible compositions includes identified or selected active compounds of the foregoing aspects, including any examples or combination of examples thereof, as set forth above, wherein the concentration of the identified or selected active compounds in the ingestible compositions may be no more than 200 ppm. In another related aspect, the disclosure provides ingestible compositions includes any identified or selected active compounds of the foregoing aspects, including any examples or combination of examples thereof, as set forth above, wherein the ingestible compositions include no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm.

In certain examples of any aspects and examples set forth herein that refer to an ingestible composition, the ingestible composition may be a non-naturally occurring product, such as a composition specifically manufactured for the production of a flavored product, such as food or beverage product.

Thus, in some examples, the compositions set forth in any of the foregoing aspects (including in any uses or methods), include an identified or selected active compound and an umami flavoring agent. In some examples, the composition further includes a vehicle. In some examples, the vehicle may be water.

In some other examples, the umami flavoring agent may be present in an amount from 10 ppm to 2000 ppm. In some examples, the umami flavoring agent may be present in an amount from 20 ppm to 1800 ppm. In some examples, the umami flavoring agent may be present in an amount from 30 ppm to 1600 ppm. In some examples, the umami flavoring agent may be present in an amount from 40 ppm to 1500 ppm. In some examples, the umami flavoring agent may be present in an amount from 50 ppm to 1400 ppm. In some examples, the umami flavoring agent may be present in an amount from 50 ppm to 1300 ppm. In some examples, the umami flavoring agent may be present in an amount from 50 ppm to 1200 ppm. In some examples, the umami flavoring agent may be present in an amount from 50 ppm to 1150 ppm. In some examples, the umami flavoring agent may be present in an amount from 50 ppm to 1100 ppm. In some examples, the umami flavoring agent may be present in an amount from 50 ppm to 1000 ppm. In some further examples of the examples set forth in this paragraph, the umami flavoring agent may be a glutamate, a glutamate salts, a ribonucleotide, a derivative of one or more of the foregoing, or any combination thereof. In some examples, the umami flavoring agent includes a glutamate salt such as monosodium glutamate (MSG), monopotassium glutamate, calcium glutamate, monoammonium glutamate, magnesium glutamate, sodium glutamate, potassium glutamate, or any combination thereof. In some examples, the umami flavoring agent includes a ribonucleotide, such as a 5′-ribonucleotide, guanosine 5′-monophosphate (GMP), inosine 5′-monophosphate (IMP), adenosine 5′monophosphate (AMP), or any combination thereof.

The umami flavoring agent may include a natural flavoring agent or synthetic natural flavoring agent that includes, but is not limited to, hydrolized vegetable protein, autolyzed yeast, yeast extract, yeast food, nutritional yeast, cheese products, savory fermented seasonings, sauce or liquid flavoring, and combinations or blends of any two or more thereof. In some examples, cheese products may include parmesan, cheddar, emmental, stilton, Roquefort, gruyere, Saint Paulin, camembert, Danish blue, gouda, cotija, chevre, feta, mozzarella, taleggio, manchego, Monterey jack, wagasi, ayibe, caravane, chechil, chhana, byaslag, chura kampo, chura loenpa, nguri, rubing, rushan, bandel, paneer, chhana, dahi chhana, kalari, kalimpon, dangke, sakura, imsil, susa masam, byaslag, flower of Rajya, chhurpi, kesong puti, djathë i bardhë, kaçkavall, djathë pice, gjizë, bachensteiner, bergkase, brimsen, Gelundener Käse, Montafoner Sauerkäse, mondseer, staazer, steirerkäse, Tyrolean grey, brussels, chimay, herve, le Wavreumont, limburger, maredsous, passendale, remoudou, rodoric, livno, Herzegovina “squeaking,” trappista, Vlas̆ić/Travnic̆ki, Bosnian smoked, cherni vit, kashkaval, sirene, Pas̆ki sir, S̆kripavac tounjski, prgica, dimsi, akkawi, anari, halloumi, kefalotyri, abertam, Blat'ácké zlato, Olomoucké syrec̆ky, hermelin, danbo, esrom, fynbo, havarti, maribo, molbo, saga, samsø, tybo, vesterhavsost, atleet, eesti juust, kadaka juust, aura, lappi, leipäjuusto, oltermanni, raejuusto, brie, munster, fromage, quark, jarlsberg, grevé, cottage cheese, labneh, halloumi, knaan, cheese curds, añejo, oaxaca, queso crema, chihuahua, queso fresco, queso panela, bergenost, colby, colby-jack, cream cheese, humboldt fog, liederkranz, pepper jack, provel, pinconning, red hawk, teleme, goya, reggianito, queijo canastra, quesode mano, farmer cheese, or any combination thereof. In some examples, savory fermented seasonings include soy sauce, worcestershire sauce, miso, kimchi, or any combination thereof. In some examples, sauce or liquid flavoring includes marmite, vegemite, fish sauce, oyster sauce, green tea, or any combination thereof.

The identified or selected active compounds can be present in the ingestible compositions in any suitable amount. In some examples, the identified or selected active compounds are present in an amount sufficient to enhance the taste, for instance, by enhancing the umami taste of the compositions. Thus, in some examples, the ingestible composition includes the identified or selected active compound in a concentration no greater than 200 ppm, or no greater than 150 ppm, or no greater than 100 ppm, or no greater than 50 ppm, or no greater than 40 ppm, or no greater than 30 ppm, or no greater than 20 ppm. In some examples, the identified or selected active compound may be present in a minimum amount, such as 1 ppm or 5 ppm. Thus, in some examples, the ingestible composition includes the identified or selected active compound in a concentration ranging from 1 ppm to 200 ppm, or from 1 ppm to 150 ppm, or from 1 ppm to 100 ppm, or from 1 ppm to 50 ppm, or from 1 ppm to 40 ppm, or from 1 ppm to 30 ppm, or from 1 ppm to 20 ppm, or from 5 ppm to 200 ppm, or from 5 ppm to 150 ppm, or from 5 ppm to 100 ppm, or from 5 ppm to 50 ppm, or from 5 ppm to 40 ppm, or from 5 ppm to 30 ppm, or from 5 ppm to 20 ppm. In examples where an umami flavoring agent, such as monosodium glutamate, may be present, the weight-to-weight ratio of flavoring agent to the identified or selected active compound in the ingestible composition ranges from 1000:1 to 50000:1, or from 1000:1 to 10000:1, or from 2000:1 to 8000:1.

The ingestible compositions can, in certain examples, include any additional ingredients or combination of ingredients as are commonly used in food and beverage products, including, but not limited to:
umami ingredients, including, for example meat broths, soy sauce, tomato sauce, cheeses, worcestershire sauce, glutamates, or ribonucleotides;
umami enhancing ingredients;
acids, including, for example citric acid, phosphoric acid, ascorbic acid, sodium acid sulfate, lactic acid, or tartaric acid;
bitter ingredients, including, for example caffeine, quinine, green tea, catechins, polyphenols, green robusta coffee extract, green coffee extract, potassium chloride, menthol, or proteins (such as proteins and protein isolates derived from plants, algae, or fungi);
coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;
preservatives, including, for example sodium benzoate, potassium benzoate, potassium sorbate, sodium metabisulfate, sorbic acid, or benzoic acid;
antioxidants including, for example ascorbic acid, calcium disodium EDTA, alpha tocopherols, mixed tocopherols, rosemary extract, grape seed extract, resveratrol, or sodium hexametaphosphate;
vitamins or functional ingredients including, for example resveratrol, Co-Q10, omega 3 fatty acids, theanine, choline chloride (citocoline), fibersol, inulin (chicory root), taurine, panax ginseng extract, guanana extract, ginger extract, L-phenylalanine, L-carnitine, L-tartrate, D-glucoronolactone, inositol, bioflavonoids, Echinacea, ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate;
clouding agents, including, for example ester gun, brominated vegetable oil (BVO), or sucrose acetate isobutyrate (SAIB);
buffers, including, for example sodium citrate, potassium citrate, or salt;
flavors, including, for example propylene glycol, ethyl alcohol, glycerine, gum Arabic (gum acacia), maltodextrin, modified corn starch, dextrose, natural flavor, natural flavor with other natural flavors (natural flavor WONF), natural and artificial flavors, artificial flavor, silicon dioxide, magnesium carbonate, or tricalcium phosphate;
starches and stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), inulin, or carrageenan; or
sweeteners, including agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, Stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, glycyrrhetic acid monoglucuronide, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside IV, mogroside V, and neomogroside), mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

The ingestible compositions may have any suitable pH. In some examples, the identified or selected active compounds enhance the umami flavor of an umami flavoring agent under a broad range of pH, e.g., from lower pH to neutral pH. The lower and neutral pH includes, but is not limited to, a pH from about 2.5 to about 8.5; from about 3.0 to about 8.0; from about 3.5 to about 7.5; and from about 4.0 to about 7. In certain examples, compounds as disclosed and described herein, individually or in combination, can enhance the perceived umami flavor of a fixed concentration of an umami flavoring agent in taste tests at a compound concentration of about 50 M, 40 M, 30 M, 20 M, or 10 M at both low to neutral pH value. In certain examples, the enhancement factor of the compounds as disclosed and described herein, individually or in combination, at the lower pH may be substantially similar to the enhancement factor of the compounds at neutral pH. Such consistent umami enhancing property under a broad range of pH allow a broad use in a wide variety of foods and beverages of the compounds as disclosed and described herein, individually or in combination.

### Flavored Products and Concentrates

In certain aspects, the disclosure provides flavored products includes any compositions of the preceding aspects. In some examples, the flavored products are beverage products, such as soda, flavored water, tea, and the like. In some other examples, the flavored products are food products, such as yogurt or ramen noodles.

In examples where the flavored product may be a beverage, the beverage may be selected from the group consisting of vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts, protein drinks, protein shakes, and smoothies. In some examples, the beverage may be a soft drink.

In certain examples of any aspects and examples set forth herein that refer to a flavored product, the flavored product may be a non-naturally occurring product, such as a packaged food or beverage product.

Further non-limiting examples of food and beverage products or formulations include umami seasoning, coatings, or glazes for such products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommé) to sauces (cream or cheese-based soups).

The Dehydrated and Culinary Food Category usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or iced tea, such as black, green, white, oolong, and flavored tea; and other drinks including flavor-, malt- or plant-based powders, granules, blocks or tablets mixed with milk or water.

The Snack Food category generally refers to any food that can be a light informal meal including, but not limited to sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; soy, oat, bean (e.g., red bean and mung bean), and rice-based ice creams.

The Confectionery category generally refers to edible product that may be sweet to the taste. Examples of confectionery include, but are not limited to candies, gelatins, chocolate confectionery, sugar confectionery, gum, and the likes and any combination products.

The Meal Replacement category generally refers to any food intended to replace the normal meals, particularly for people having health or fitness concerns. Examples of meal replacement include, but are not limited to, slimming products and convalescence products.

The Ready Meal category generally refers to any food that can be served as meal without extensive preparation or processing. The ready meal includes products for which the manufacturer has already performed many of the steps to prepare the food before shipping to consumers, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to, canned/preserved meat and meat products, fish/seafood, vegetables, tomatoes, beans, fruit, ready meals, soup, pasta, and other canned/preserved foods.

The Frozen Processed Food category includes, but is not limited to, frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to, rice, dessert mixes, dried ready meals, dehydrated soup, instant soup, dried pasta, plain noodles, and instant noodles.

The Chill Processed Food category includes, but is not limited to, chilled processed meats, processed fish/seafood products, lunch kits, fresh cut fruits, ready meals, pizza, prepared salads, soup, fresh pasta and noodles.

The Sauces, Dressings and Condiments category includes, but is not limited to, tomato pastes and purees, bouillon/stock cubes, herbs and spices, monosodium glutamate (MSG), table sauces, soy-based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to, milk- or soybean-based formula; and prepared, dried and other baby food.

The Spreads category includes, but is not limited to, jams and preserves, honey, chocolate spreads, nut-based spreads, and yeast-based spreads.

The Dairy Product category generally refers to edible product produced from mammal's milk. Examples of dairy product include, but are not limited to drinking milk products, cheese, yoghurt and sour milk drinks, and other dairy products.

Additional examples for flavored products, particularly food and beverage products or formulations, are provided as follows. Exemplary ingestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, ready-to-eat cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/ultra-high temperature (UHT) milk, full fat long life/UHT milk, semi skimmed long life/UHT milk, fat-free long life/UHT milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy-based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads. Exemplary ingestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof. Exemplary ingestible compositions also include ramen noodles, savory solid or liquid concentrate compositions for preparing soups or sauces, ideally so as to enable the reduction in concentration of previously known umami additives, or artificial umami flavoring agents.

In some examples, compounds as disclosed and described herein, individually or in combination, enhance the umami taste or other taste properties of other natural or synthetic tastants, and ingestible compositions made therefrom. In one example, the compounds as disclosed and described herein, individually or in combination, may be used or provided in its umami tastant enhancing concentration(s). For example, the compounds as disclosed and described herein, individually or in combination, may be present in an amount of from 0.001 ppm to 100 ppm, or narrower alternative ranges from 0.1 ppm to 50 ppm, from 0.01 ppm to 40 ppm, from 0.05 ppm to 30 ppm, from 0.01 ppm to 25 ppm, or from 0.1 ppm to 30 ppm, or from 0.1 ppm to 25 ppm, or from 1 ppm to 30 ppm, or from 1 ppm to 25 ppm.

In some examples, identified or selected active compounds as disclosed and described herein, individually or in combination, may be provided in a flavoring concentrate formulation, e.g., suitable for subsequent processing to produce a ready-to-use (i.e., ready-to-serve) product. By “a flavoring concentrate formulation,” it is meant a formulation which should be reconstituted with one or more diluting medium to become a ready-to-use composition. The term “ready-to-use composition” may be used herein interchangeably with “ingestible composition,” which denotes any substance that, either alone or together with another substance, can be taken by mouth whether intended for consumption or not. In one example, the ready-to-use composition includes a composition that can be directly consumed by a human or animal. The flavoring concentrate formulation is typically used by mixing with or diluted by one or more diluting medium, e.g., any consumable or ingestible ingredient or product, to impart or modify one or more flavors to the diluting medium. Such a use process is often referred to as reconstitution. The reconstitution can be conducted in a household setting or an industrial setting. For example, a frozen fruit juice concentrate can be reconstituted with water or other aqueous medium by a consumer in a kitchen to obtain the ready-to-use fruit juice beverage. In another example, a soft drink syrup concentrate can be reconstituted with water or other aqueous medium by a manufacturer in large industrial scales to produce the ready-to-use soft drinks. In another example, a bouillon cube can be reconstituted with water or other aqueous medium by a consumer in a kitchen to obtain the ready-to-use soup broth. Since the flavoring concentrate formulation has the flavoring agent or flavor modifying agent in a concentration higher than the ready-to-use composition, the flavoring concentrate formulation is typically not suitable for being consumed directly without reconstitution. There are many benefits of using and producing a flavoring concentrate formulation. For example, one benefit may be the reduction in weight and volume for transportation as the flavoring concentrate formulation can be reconstituted at the time of usage by the addition of suitable solvent, solid or liquid.

The flavored products set forth according to any of the foregoing examples, also include, in certain examples, one or more additional flavor-modifying compounds, such as compounds that enhance umami (e.g., MSG, ribonucleotides, etc.), compounds that block umami, compounds that enhance sweetness, compounds that block bitterness, compounds that reduce sourness, compounds that enhance saltiness, compounds that enhance a cooling effect, or any combinations of the foregoing.

In certain examples of any aspects and examples set forth herein that refer to a flavoring concentrate, the flavoring concentrate may be a non-naturally occurring product, such as a composition specifically manufactured for the production of a flavored product, such as food or beverage product.

In one example, the flavoring concentrate formulation includes i) compounds as disclosed and described herein, individually or in combination; ii) a carrier; and iii) optionally at least one adjuvant. The term “carrier” denotes a usually inactive accessory substance, such as solvents, binders, or other inert medium, which may be used in combination with the present compound and one or more optional adjuvants to form the formulation. For example, water or starch can be a carrier for a flavoring concentrate formulation. In some examples, the carrier may be the same as the diluting medium for reconstituting the flavoring concentrate formulation; and in other examples, the carrier may be different from the diluting medium. The term “carrier” as used herein includes, but is not limited to, ingestibly acceptable carrier.

The term “adjuvant” denotes an additive which supplements, stabilizes, maintains, or enhances the intended function or effectiveness of the active ingredient, such as a tastant. In one example, the at least one adjuvant includes one or more flavoring agents. The flavoring agent may be of any flavor known to one skilled in the art or consumers, such as the flavor of chocolate, coffee, tea, mocha, French vanilla, peanut butter, chai, or combinations thereof. In another example, the at least one adjuvant includes one or more umami taste enhancer, identified as described above. In another example, the at least one adjuvant includes one or more ingredients selected from the group consisting of an emulsifier, a stabilizer, an antimicrobial preservative, an antioxidant, vitamins, minerals, fats, starches, protein concentrates and isolates, salts, and combinations thereof. Examples of emulsifiers, stabilizers, antimicrobial preservatives, antioxidants, vitamins, minerals, fats, starches, protein concentrates and isolates, and salts are described in U.S. Pat. No. 6,468,576, the content of which is hereby incorporated by reference in its entirety for all purposes.

In one example, the flavoring concentrate formulation may be formulated to have a low water activity for extended shelf life. Water activity is the ratio of the vapor pressure of water in a formulation to the vapor pressure of pure water at the same temperature. In one example, the flavoring concentrate formulation has a water activity of less than about 0.85. In another example, the flavoring concentrate formulation has a water activity of less than about 0.80. In another example, the flavoring concentrate formulation has a water activity of less than about 0.75.

In one example, the flavoring concentrate formulation has the present compound in a concentration that is at least 2 times of the concentration of the compound in a ready-to-use composition. In one example, the flavoring concentrate formulation has the present compound in a concentration that is at least 5 times of the concentration of the compound in a ready-to-use composition. In one example, the flavoring concentrate formulation has the present compound in a concentration that is at least 10 times of the concentration of the compound in a ready-to-use composition. In one example, the flavoring concentrate formulation has the present compound in a concentration that is at least 15 times of the concentration of the compound in a ready-to-use composition. In one example, the flavoring concentrate formulation has the present compound in a concentration that is at least 20 times of the concentration of the compound in a ready-to-use composition. In one example, the flavoring concentrate formulation has the present compound in a concentration that is at least 30 times of the concentration of the compound in a ready-to-use composition. In one example, the flavoring concentrate formulation has the present compound in a concentration that is at least 40 times of the concentration of the compound in a ready-to-use composition. In one example, the flavoring concentrate formulation has the present compound in a concentration that is at least 50 times of the concentration of the compound in a ready-to-use composition. In one example, the flavoring concentrate formulation has the present compound in a concentration that is at least 60 times of the concentration of the compound in a ready-to-use composition. In one example, the flavoring concentrate formulation has the present compound in a concentration that is up to 100 times of the concentration of the compound in a ready-to-use composition.

### Terminology

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. The terms “comprising,” “including,” “having,” and the like are synonymous and are used inclusively, in an open-ended fashion, and do not exclude additional elements, features, acts, operations, and so forth. That is, the above terms are to be interpreted synonymously with the phrases “having at least” or “including at least.” For example, when used in the context of a process, the term “comprising” means that the process includes at least the recited steps, but may include additional steps. When used in the context of a device, the term “comprising” means that the device includes at least the recited features or components, but may also include additional features or components. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

As used herein, “solvate” means a compound formed by the interaction of one or more solvent molecules and one or more compounds described herein. In some examples, the solvates are ingestibly acceptable solvates, such as hydrates.

An “umami compound” refers to a compound that elicits a detectable umami flavor in a subject, e.g., a compound that activates a T1R1+3 receptor in vitro. An “umami receptor blocker” or “umami blocker” refers to a compound that antagonizes the activation the T1R1+3 receptor in vitro by an umami compound.

A “umami additive,” “umami flavoring agent,” “umami flavor entity,” or “umami compound” herein refers to a compound or ingestibly acceptable salt thereof that elicits a detectable umami flavor in a subject, e.g., a compound that activates and/or modulates the T1R1+3 receptor in vitro.

As used herein, “T1R1 umami taste receptor subunit,” “T1R1 umami taste receptor,” “T1R1 taste receptor,” “T1R1 receptor,” “T1R1 subunit,” “T1R1,” “umami taste receptor,” and “umami receptor” are all equivalent terms that may refer to the T1R1 umami taste receptor subunit, which is a polypeptide that may sense the presence of umami compounds when dimerized with a T1R3 taste receptor subunit.

As used herein, “rat-human T1R3 taste receptor subunit,” “rat-human T1R3 taste receptor,” “rat-human T1R3,” “chimeric rat-human T1R3 taste receptor subunit,” “rat-human chimeric T1R3 taste receptor subunit,” “chimeric rat-human T1R3 taste receptor,” “rat-human chimeric T1R3 taste receptor,” “chimeric T1R3 taste receptor subunit,” “chimeric T1R3 receptor subunit,” “chimeric T1R3 receptor,” and “chimeric T1R3” are all equivalent terms that may refer to the rat-human T1R3 taste receptor subunit, which is a polypeptide including a portion of a human T1R3 subunit and a portion of a rat T1R3 subunit that is capable of dimerizing with a T1R1 taste receptor subunit and may enable the T1R1 umami taste receptor subunit to interact with a G-protein to increase intracellular Ca²⁺ levels when T1R1 senses an umami compound.

As used herein, “T1R1+3 heterodimer,” “T1R1+3 dimer,” “T1R1+3 receptor,” “T1R1+3” are all equivalent terms that may refer to the dimer of T1R1 and T1R3.

Conditional language, such as “can,” “could,” “might,” or “may,” unless specifically stated otherwise, or otherwise understood within the context as used, is generally intended to convey that certain examples include, while other examples do not include, certain features, elements, or steps. Thus, such conditional language is not generally intended to imply that features, elements, or steps are in any way required for one or more examples or that one or more examples necessarily include logic for deciding, with or without user input or prompting, whether these features, elements, or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount.

The term “and/or” as used herein has its broadest least limiting meaning, which is the disclosure includes A alone, B alone, both A and B together, or A or B alternatively, but does not require both A and B or require one of A or one of B. As used herein, the phrase “at least one of” A, B, “and” C should be construed to mean a logical A or B or C, using a non-exclusive logical or.

Conditional language used herein, such as, among others, “can,” “could,” “might,” “may,” “e.g.,” and the like, unless specifically stated otherwise, or otherwise understood within the context as used, is generally intended to convey that certain, certain features, elements and/or steps are optional. Thus, such conditional language is not generally intended to imply that features, elements and/or steps are in any way required or that one or more implementations necessarily include logic for deciding, with or without other input or prompting, whether these features, elements and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Any methods disclosed herein need not be performed in the order recited. The methods disclosed herein include certain actions taken by a practitioner; however, they can also include any third-party instruction of those actions, either expressly or by implication.

### EXAMPLES

### Example 1—Dose Response of U2OS Cells

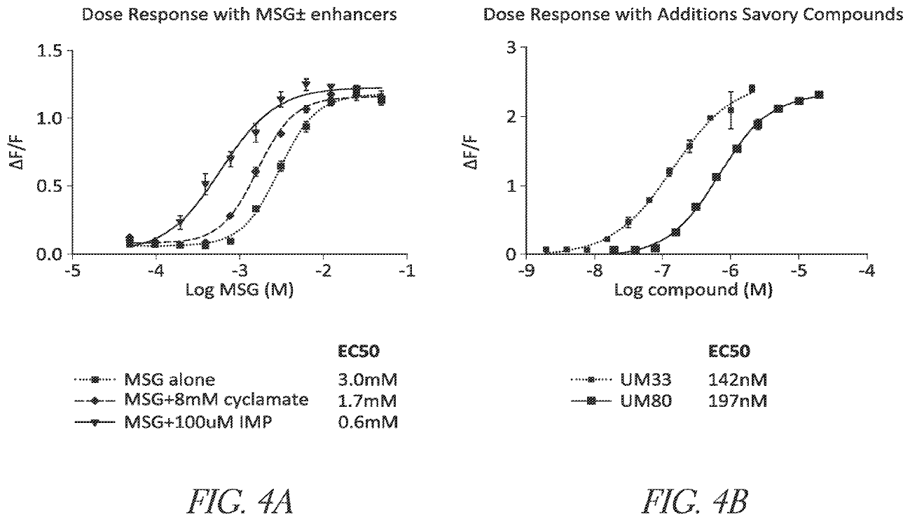

A stable cell line expressing a human T1R1 (SEQ ID NO. 1), a rat-human T1R3 chimera (SEQ ID NO. 3), and the promiscuous G-protein G16gust25 was generated in U2OS cells and characterized in assays in which the cell line was exposed to savory (umami) compounds and umami enhancers. FIG. 4A shows the response of this cell line to MSG, an umami tastant, with or without known umami enhancers cyclamate or IMP. The x-axis plots the log of MSG concentration, while the y-axis plots fold change in fluorescence of a Ca²⁺-sensitive fluorescent dye in response to intracellular Ca²⁺ concentration. Cyclamate was added at a concentration of 8 mM while IMP was added at a concentration of 100 M. FIG. 4A also indicates the effective concentrations of MSG necessary to provoke 50% or the maximal response (EC50). Notably, the fluorescence detectably increases as MSG concentration increases, which is expected for a cell line including a functional T1R1+3 umami taste receptor subunit within a T1R1+3 dimer coupled to a G-protein. The addition of either umami enhancer reduced the concentration of MSG necessary to reach the EC50 threshold.

FIG. 4B illustrates dose response assays of the stable U2OS cell line expressing a human T1R1 (SEQ ID NO. 1), a rat-human T1R3 chimera (SEQ ID NO. 3), and the promiscuous G-protein G16gust25 in response to the addition of high-potency umami tastants and enhancer compounds UM33 or UM80. UM33 and UM88 have the following structures and are described in PCT Publication No. WO 2005/041684 (incorporated herein by reference in its entirety):

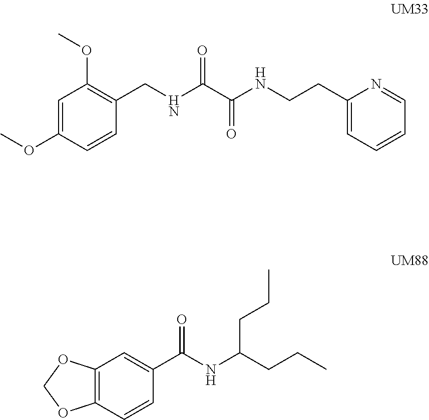 [SMILES: COc1ccc(CNC(=O)C(=O)NCCc2ccccn2)c(OC)c1 ; CCCC(CCC)NC(=O)c1ccc2c(c1)OCO2]

The x-axis plots the log of savory compound concentration, while the y-axis plots fold change in fluorescence of a Ca²⁺-sensitive fluorescent dye in response to intracellular Ca²⁺ concentration. FIG. 4A also indicates the EC50 for each compound. Notably, the fluorescence detectably increases as umami enhancer compound concentration increases, which is expected for a cell line including a functional T1R1+3 umami taste receptor subunit, a rat-human T1R3 taste receptor subunit, and a coupled G-protein.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain implementations disclosed herein is indicated by the appended claims rather than by the foregoing description. All changes which come within the meaning and range of equivalency of the claims are to be embraced within their scope.

1. A cell comprising
a T1R1+3 taste receptor comprising
a T1R1 umami taste receptor subunit,
a rat-human T1R3 taste receptor subunit; and
a G-protein,
wherein the cell is not an HEK cell.

2.-35. (canceled)

36. The cell of claim 1, wherein the cell is a eukaryotic cell.

37. The cell of claim 1, wherein the cell is a U2OS cell.

38. The cell of claim 1, wherein the G-protein is coupled to the T1R1 umami taste receptor subunit.

39. The cell of claim 1, wherein the T1R1 umami taste receptor subunit comprises a polypeptide sequence at least 80% identical to a polypeptide sequence of SEQ ID NO. 1, or a functional fragment thereof.

40. The cell of claim 1, wherein the T1R1 umami taste receptor subunit comprises a polypeptide sequence at least 95% identical to a polypeptide sequence of SEQ ID NO. 1, or a functional fragment thereof.

41. The cell of claim 1, wherein the rat-human T1R3 taste receptor subunit comprises a polypeptide sequence at least 80% identical to a polypeptide sequence of SEQ ID NO. 3, or a functional fragment thereof.

42. The cell of claim 1, wherein the rat-human T1R3 taste receptor subunit comprises a polypeptide sequence at least 95% identical to a polypeptide sequence of SEQ ID NO. 3, or a functional fragment thereof.

43. The cell of claim 1, wherein the G-protein comprises a promiscuous G-protein, wherein the promiscuous G-protein comprises any one of the following: G15, G16, G16T44, or G16gust25.

44. The cell of claim 1, wherein the G-protein comprises a polypeptide sequence at least 90% identical to a polypeptide sequence of SEQ ID NO: 5, or a functional fragment thereof.

45. The cell of claim 1, wherein the cell comprises a polynucleotide sequence encoding for the T1R3 taste receptor subunit comprising a sequence at least 80% identical to a polynucleotide sequence of SEQ ID NO. 4, or a functional fragment thereof.

46. The cell of claim 1, wherein the cell comprises a polynucleotide sequence encoding for the T1R3 taste receptor subunit comprising a sequence at least 95% identical to a polynucleotide sequence of SEQ ID NO. 4, or a functional fragment thereof.

47. The cell of claim 1, wherein the cell is modified to overexpress the T1R1 umami taste receptor subunit.

48. The cell of claim 1, wherein the cell is modified to overexpress the rat-human T1R3 taste receptor subunit.

49. The cell of claim 1, wherein the cell is modified to overexpress the G-protein.

50. A method of identifying an umami taste modulator, the method comprising
contacting the cell of claim 1 with a test compound; and
measuring a T1R1+3 umami taste receptor activity, wherein a change in the T1R1+3 umami taste receptor activity indicates that the test compound is an umami taste modulator.

51. The method of claim 50, wherein measuring the T1R1+3 umami taste receptor activity comprises detecting changes in intracellular calcium.

52. The method of claim 51, wherein detecting changes in intracellular calcium comprises detecting a Fluo-4 dependent fluorescence.

53. The method of claim 52, wherein measuring the T1R1+3 umami taste receptor activity comprises comparing the T1R1+3 umami taste receptor activity in presence of the test compound and in absence of the test compound.

54. The method of claim 53, wherein measuring the T1R1+3 umami taste receptor activity comprises comparing the T1R1+3 umami taste receptor activity in presence of a known umami tastant and in absence of the known umami tastant.
